SEQUENCE LISTING (7 sequences)

SEQ ID NO: 1 - protein, 94 aa, Homo sapiens
  ref <301>: AUTHORS: Ulvsback, M., Lindstrom, C., Weiber, H., Abrahamson, P.A., Lilja, H., and Lundwall, A“
  ref <302>: TITLE: Molecular cloning of a small prostate protein, known as beta-microsemenoprotein, PSP94 or beta-inhibin, and demonstration of transcripts in non-genital tissues.
  ref <303>: JOURNAL: Biochem. Biophys. Res Commun.
  ref <304>: VOLUME: 164
  ref <305>: ISSUE: 3
  ref <306>: PAGES: 1310-1315
  ref <307>: DATE: 1989
  ref <308>: DATABASE ACCESSION NUMBER: GI 131436
  ref <309>: DATABASE ENTRY DATE: 1988-08-01
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 102 aa, Artificial Sequence
  recombinant human PSP94 (rHuPSP94) produced from yeast
EAEAYVEFSCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - protein, 10 aa, Artificial Sequence
  decapeptide
YTCSVSEPGI

SEQ ID NO: 4 - protein, 15 aa, Artificial Sequence
  Polypeptide 7-21
NEGVPGDSTRKCMDL

SEQ ID NO: 5 - protein, 15 aa, Artificial Sequence
  PCK3145 (polypeptide 31-45)
EWQTDNCETCTCYET

SEQ ID NO: 6 - protein, 19 aa, Artificial Sequence
  Polypeptide 76-94
IVVEKKDPKKTCSVSEWII

SEQ ID NO: 7 - protein, 15 aa, Artificial
  polypeptide derived from PCK3145 sequence (polypeptide analog)
  MISC_FEATURE (1)..(1): Xaa may be glutamic acid, asparagine or aspartic acid
  MISC_FEATURE (4)..(4): Xaa may be threonine or serine
  MISC_FEATURE (6)..(6): Xaa may be glutamic acid, asparagine or aspartic acid
  MISC_FEATURE (8)..(8): Xaa may be glutamic acid, asparagine or aspartic acid
  MISC_FEATURE (9)..(9): Xaa may be threonine or serine
  MISC_FEATURE (11)..(11): Xaa may be threonine or serine
  MISC_FEATURE (13)..(13): Xaa may be may be tyrosine or phenylalanine
  MISC_FEATURE (14)..(14): Xaa may be glutamic acid, asparagine or aspartic acid
  MISC_FEATURE (15)..(15): Xaa may be threonine or serine
XWQXDXCXXCXCXXX

(19) United States
(12) Patent Application Publication
(10) Pub. No.: US 2005/0026833 A1
(43) Pub. Date: Feb. 3, 2005

(54) PSP-94: use for treatment of hypercalcemia and bone metastasis

(76) Inventors: Shafaat Ahmed Rabbani, Westmount (CA); Nicholas Shukeir, Montreal (CA); Chandra J. Panchal, London (CA); Christopher Newman, Beaconsfield (CA)

Correspondence Address: BROUILLETTE KOSIE PRINCE, 25th Floor, 1100 Rene-Levesque Blvd. West, Montreal, QC, H3B 5C9, CA

(21) Appl. No.: 10/857,358

(22) Filed: Jun. 1, 2004

Related U.S. Application Data
(63) Continuation-in-part of application No. 10/291,360, filed on Nov. 8, 2002.

(30) Foreign Application Priority Data
Nov. 8, 2001 (CA) 2,361,736

(51) Int. Cl. A61K 38/17; C07K 14/47

(52) U.S. Cl. 514/12; 530/350

(57) ABSTRACT

The present invention discloses the use of PSP-94, PCK3145 and other derivatives and biologically active analogues for treating a patient having a condition such as metastasis, metastatic cancer, a condition associated with elevated levels of parathyroid hormone-related protein (PTHrP), PTHrP-induced osteolysis and/or hypercalcemia of malignancy. These compounds were found to be effective treatment modalities for bone metastasis caused by prostate cancer. Furthermore, decrease in cellular and plasma PTHrP levels as well as plasma calcium levels observed by treatment with such compound can serve as useful biochemical markers for monitoring the efficacy of these anti-metastatic compounds.

This application is a continuation-in-part of U.S. patent application Ser. No.:10/291,360, filed on Nov. 8, 2002, the entire content of which is incorporated herein by reference.

### FIELD OF THE INVENTION

The present invention relates to compounds, pharmaceutical compositions and method for treating patients with metastasis, metastatic cancer, a condition associated with elevated levels of parathyroid hormone-related protein (PTHrP), PTHrP-induced osteolysis and/or hypercalcemia of malignancy. More particularly, the present invention relates to the use of SEQ ID NO.:1, SEQ ID NO.:2, SEQ ID NO.:5, SEQ ID NO.:1 analogue, SEQ ID NO.:2 analogue, SEQ ID NO.:5 analogue (e.g., SEQ ID NO.:7) and other PSP94 derivatives or fragments such as for example, polypeptide 7-21 (SEQ ID NO.:4), the decapeptide (SEQ ID NO.:3), the polypeptide 76-94 (SEQ ID NO.:6) and their biologically active analogues, as well as combination thereof for treating such conditions.

### BACKGROUND OF THE INVENTION

The prostate gland, which is found exclusively in male mammals, produces several components of semen and blood and several regulatory peptides. The prostate gland comprises stroma and epithelium cells, the latter group consisting of columnar secretory cells and basal nonsecretory cells. A proliferation of these basal cells as well as stroma cells gives rise to benign prostatic hyperplasia (BPH), which is one common prostate disease. Another common prostate disease is prostatic adenocarcinoma (CaP), which is the most common of the fatal pathophysiological prostate cancers, and involves a malignant transformation of epithelial cells in the peripheral region of the prostate gland. Prostatic adenocarcinoma and benign prostatic hyperplasia are two common prostate diseases, which have a high rate of incidence in the aging human male population. Approximately one out of every four males above the age of 55 suffers from a prostate disease of some form or another. Prostate cancer is the second most common cause of cancer related death in elderly men, with approximately 96,000 cases diagnosed and about 26,000 deaths reported annually in the United States. A distinct feature of prostate cancer is its ability to cause osteoblastic skeletal metastasis which contributes to the high rate of morbidity and mortality associated with this hormone dependent malignancy. Skeletal metastases is often associated with hormone refractory prostate cancer. A major cause of prostate cancer morbidity is bone pain, a result of nerve impingement by skeletal metastatic tumors. An essential component in cancer therapeutics is palliative care and improved quality of life in patients with late stage cancer. As such the ability to eliminate or reduce bone pain would be of imperial value.

Hypercalcemia has been recognized as a complication of malignancy since 1920 and occurs in at least 15-20% of patients harbouring a variety of cancers including prostate cancer. Although no single agent has been shown to be uniquely responsible for the hypercalcemia of malignancy (HM), increased production of parathyroid hormone related peptide (PTHrP) by tumor cells has led to its establishment as the major pathogenic factor responsible for HM. This is of particular significance in prostate and breast cancer which are often associated with skeletal metastasis where osteolytic effects of PTHrP results in increased bone resorption and hypercalcemia.

Clinical prostate cancer can be treated successfully at its early stage when the cancer is well confined within the prostate gland. However, increased production of many factors including growth factors, sex steroids, angiogenic factors and proteases such as urokinase (uPA) and matrix metalloproteinases (MMPs) by tumor cells and their surrounding stroma is associated with high mortality. Despite recent advances in the therapeutic modalities for organ confined prostate cancer including surgery and radiotherapy, limited success has been obtained in treating hormone-independent metastatic prostate cancer. It is generally accepted that cancer cells that metastasize to distant organs exhibit organ-specific characteristics that are often distinct from that of the primary tumor (Fidler, I. J., J. Natl. Cancer Inst. 87(21):1588-92, 1995). For example, cancer cells with a high predilection to metastasize to bone therefore must have properties not present in tumors that rarely metastasize to bone (Boyce, B. F., et al., Endocr. Relat. Cancer. 6(3):333-47, 1999). Therapies which may have an inhibitory effect on the growth of primary tumor cells will not necessarily have a similar effect on metastatic cancer cells. Therefore, therapies specifically targeting metastatic cancer cells must be developed.

Prostate specific antigen (PSA) and prostate secretory protein of 94 amino acids (herein referred to PSP-94 or PSP) are known to serve as prognostic markers for disease progression. Like PSA, PSP-94 levels in serum, urine, and prostate tissue of patients with prostate cancer are inversely related to tumor grade. In previous work, described in U.S. Pat. No. 5,428,011 (the entire content of which is incorporated herein by reference), pharmaceutical preparations (i.e., compositions) of native human seminal plasma PSP-94 (SEQ ID NO.:1) were provided for inhibiting in-vitro and in-vivo cancerous prostate, gastrointestinal and breast tumors. In additional work disclosed in Canadian patent application No: 2,359,650 (the entire content of which is incorporated herein by reference), the ability of PSP-94 and PSP-94 fragments, such as PCK3145, to be used in the inhibition of tumor growth and more particularly, in the inhibition of prostate cancer tumor growth was illustrated.

In the present study, the effect of PSP-94, PSP-94 derivatives, fragments and analogs, on metastatic cancer cell (e.g., skeletal metastasis, metastatic prostate cancer) was evaluated. For these studies syngeneic in vivo model of rat prostate cancer using the rat prostate cancer cell line Dunning R3227 Mat Ly Lu transfected with the full length cDNA encoding rat PTHrP was used (Rabbani, S. A. et al., Int. J. Cancer, 80: 257-264,1999). Following sub-cutaneous (S.C.) or intracardiac (I.C.) inoculation of Mat Ly Lu-PTHrP cells, the ability of different doses of PSP-94 PSP-94 derivatives, fragments or analogs to reduce tumor growth, metastasis, tumoral PTHrP production, plasma calcium and plasma PTHrP was evaluated.

Amino acid sequence homology between human and rat PSP and similarity in their tertiary structures as determined by highly conserved cysteine residues has allowed the use of human PSP-94 for these studies (Fernlund, P. et al., Arch. Biochem.Biophys. 334:73-82,1996). Due to the high levels of PTHrP production these animals routinely develop hypercalcemia, a common complication in many patients suffering from prostate cancer (Iwamura, M., et al., Urology, 43: 675-679,1994; Iwamura, M. et al., Hum. Pathol. 26: 797-801,1995). Use of this homologous model for prostate cancer allows for full interaction between the host environment and growth factors (EGF, TGF-β) (Helawell, G. O. et al., BJU Int. 89:230-240, 2002) and proteases (uPA, MMPs) (Rabbani, S. A., et al., Int. J. Cancer, 87: 276-282, 2000, Rabbani, S. A., et al., In vivo, 12:135-142, 1998) secreted by tumor cells. These prostate cancer cells are hormone-independent allowing for the evaluation of the effect of PSP-94 on late stage prostate cancer.

### SUMMARY OF THE INVENTION

The invention disclosed herein provides pharmaceutical compositions and method for treating patients with metastasis, metastatic cancer, (e.g., skeletal metastasis, metastatic prostate cancer), a condition associated with elevated levels of parathyroid hormone-related protein (PTHrP), PTHrP-induced osteolysis and/or hypercalcemia of malignancy. PSP-94 (native PSP-94 (nPSP-94) (SEQ ID NO.1)) and rHuPSP94 (recombinant human PSP-94 (SEQ ID No.2)) as well as derivatives (fragments) such as for example the decapeptide as set forth in SEQ ID NO: 3, the polypeptide as set forth in SEQ ID NO: 4 (polypeptide 7-21), the polypeptide as set forth in SEQ ID NO: 5 (PCK3145), the polypeptide as set forth in SEQ ID NO: 6 (polypeptide 76-94), the polypeptide set forth in SEQ ID NO.:7 and different polypeptide analogues of PSP-94 and or the derivatives are used herein to treat conditions related to metastasis (e.g., skeletal metastasis), metastatic cancer, a condition associated with elevated levels of parathyroid hormone-related protein (PTHrP), PTHrP-induced osteolysis and hypercalcemia of malignancy. Calcium may also be used herein as a surrogate marker of the efficacy of PSP-94 (tumor) treatment. The present invention provides, in one aspect, a method for treating a mammal (e.g.,person, patient) having a condition selected from the group consisting of metastasis, metastatic cancer, a condition associated with elevated levels of parathyroid hormone-related protein (PTHrP), PTHrP-induced osteolysis and hypercalcemia of malignancy. For example, methods of/for treating a patient suffering from hypercalcemia of malignancy may comprise administering to the patient a pharmaceutical composition comprising PSP-94, the polypeptides mentioned herein (PCK3145) and/or analogues and/or derivatives and combination thereof as defined herein.

More particularly, the method may comprise administering to the patient a compound (e.g., a pharmaceutical composition comprising a compound) which may be selected, for example, from the group consisting of SEQ ID NO.:1, SEQ ID, NO.:2, SEQ ID NO.: 3, SEQ ID NO.: 4, SEQ ID NO.:5, SEQ ID NO.:6, biologically active analogues (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6), derivatives, fragments, etc. and combinations thereof.

For example, the present invention provide the use of PSP-94, PCK3145 and analogues thereof for treating a patient (with a malignancy) suffering from hypercalcemia of malignancy (i.e., for treating hypercalcemia of malignancy) and/or to reduce (treat a patient with) hypercalcemia (related to) of malignancy and/or for reducing (lowering) calcium levels in a patient suffering from hypercalcemia of malignancy and/or to prevent occurrence of hypercalcemia of malignancy and/or to control the induction (onset) of hypercalcemia in a patient and/or to prevent (control) PTHrP increase in a patient and/or to reduce (for reducing/lowering) the level (biosynthesis, expression, transcription, translation, production, secretion) or activity of PTHrP in a patient in need thereof thereof and/or to reduce the production of agents responsible for the development of (an hypercalcemic condition) hypercalcemia including P THrP and/or reduce (delay) the development of skeletal metastasis and/or to block (reduce, impair, delay) the development (progression) of skeletal metastasis and/or to control the level of molecules involved in calcium production, wherein the molecules are selected from the group consisting of vitamine B, calcitonine and biological equivalents thereof.

In accordance with the present invention, the malignancy may be a hormone-independent malignancy. It is to be understood herein that hypercalcemia of malignancy may arise from various source including prostate cancer, breast cancer, lung carcinoma, hepatocellular carcinoma, myeloma etc. Therefore, treatment of a patient having hypercalcemia of malignancy with compounds and analogues described herein may be suitable for patient having prostate cancer, breast cancer, lung carcinoma or hepatocellular carcinoma, etc. It is therefore, to be understood herein that treatment of hypercalcemia of malignancy is not restricted to any type of malignancy.

The present invention also provides a compound selected, for example, from the group consisting of SEQ ID NO.:1, SEQ ID NO.:2, SEQ ID NO.: 3, SEQ ID NO.: 4, SEQ ID NO.:5, SEQ ID NO.:6, biologically active analogues (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combinations thereof for use in the treatment of a patient having a condition which may be selected from the group consisting of metastasis, metastatic cancer, a condition associated with elevated levels of parathyroid hormone-related protein (PTHrP), PTHrP-induced osteolysis and hypercalcemia of malignancy.

The present invention further provides the use of a compound selected, for example, from the group consisting of SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof. in the manufacture of a medicament for the treatment of a patient having a condition which may be selected from the group consisting of metastasis, metastatic cancer, a condition associated with elevated levels of parathyroid hormone-related protein (PTHrP), PTHrP-induced osteolysis and hypercalcemia of malignancy.

The present invention further provides a pharmaceutical composition which may be used in the treatment of a condition selected, for example, from the group consisting of metastasis, metastatic cancer, a condition associated with elevated levels of parathyroid hormone-related protein (PTHrP), PTHrP-induced osteolysis and hypercalcemia of malignancy, the composition may comprise a compound as defined herein (e.g., SEQ ID NO.:1, SEQ ID NO.:2, SEQ ID NO.: 3, SEQ ID NO.: 4, SEQ ID NO.:5, SEQ ID NO.:6, biologicallyactive analogue (of anyone of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof) and a pharmaceutically acceptable carrier.

The present invention also provides a method for reducing the levels of PTHrP in a mammalian cell, the method may comprise, for example, contacting the mammalian cell (directly or indirectly) with a compound as defined herein and which may be selected, for example, from the group consisting of SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.: 1, SEQ ID NO.:2, SEQ ID NO.: 3, SEQ ID NO.: 4, SEQ ID NO.:5 (e.g., SEQ ID NO.:7), SEQ ID NO.:6) and combination thereof.

The present invention further provides a method of manufacturing a medicament or a pharmaceutical composition, for the treatment of a patient having a condition selected, for example, from the group consisting of a metastasis, metastatic cancer, a condition associated with elevated levels of parathyroid hormone-related protein (PTHrP), PTHrP-induced osteolysis and hypercalcemia of malignancy, the method may comprise the steps of;
obtaining (getting, buying, borrowing, gathering, acquiring etc,) a compound as defined herein and which may be selected, for example, from the group consisting of SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof, and;
combining the compound with a pharmaceutically acceptable carrier or excipient.

The present invention also provides in one aspect thereof, a method for evaluating the efficacy of a treatment of a patient having a tumor and/or a condition which may be selected, for example, from the group consisting of bone metastasis, metastatic cancer (e.g.,to the bone), elevated levels of parathyroid hormone-related protein (PTHrP), PTHrP-induced osteolysis and hypercalcemia of malignancy, the treatment of a patient being a treatment with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof, the method comprise measuring a plasma calcium level of the patient after the treatment.

According to the present invention, the method may also comprise a step where the plasma calcium level of the patient after the treatment is compared with a plasma calcium level of the patient before the treatment. In accordance with the present invention, the plasma levels may be measured from a plasma sample of a patient.

For example, the method for evaluating, in a patient, the efficacy of the above-mentioned treatment, may comprise the steps of;
a) measuring plasma calcium from a patient with a tumor or with hypercalcemia of malignancy before the patient's treatment with a compound of the present invention,
b) measuring plasma calcium from a patient with a tumor or with hypercalcemia of malignancy after the patient's treatment with a compound of the present invention; and
c) comparing values obtained in step a) with values obtained in step b).

In another aspect, the present invention relates to an additional method for evaluating the efficacy of a treatment of a patient having a tumor and/or a condition which may be selected, for example, from the group consisting of bone metastasis, metastatic cancer (e.g.,to the bone), elevated levels of parathyroid hormone-related protein (PTHrP), PTHrP-induced osteolysis and hypercalcemia of malignancy, the treatment of a patient being a treatment with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogue (of anyone of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof, the method comprise measuring the plasma PTHrP levels of the patient.

For example, the method may comprise;
a) measuring plasma PTHrP (levels) from a patient (e.g., having a tumor or with hypercalcemia of malignancy, etc.) before his (the patient's) treatment with a compound mentioned herein,
b) measuring plasma PTHrP (levels) from the same patient after his (the patient's) treatment with a compound of the present invention; and
c) comparing values obtained in step a) with values obtained in step b).

It is to be understood herein that measurement of the cellular level of PTHrP (protein, RNA, hnRNA) is also relevant in evaluating the efficacy of an above-mentioned treatment. Measurement of cellular levels of a polypeptide may be effected by various methods known in the art, such as for example, immunological methods (e.g., immunohistochemistry, immuno-blot, enzyme-linked immunosorbent assays (ELISA)). Measurement of cellular levels of a polynucleotide such as RNA may be effected by various methods known in the art, such as for example using hybridization techniques (northern blot), polymerase chain reaction-based techniques (reverse-transcription-PCR), etc.

The present invention provides in a further aspect thereof, a method for reducing tumor cell invasion or spreading of a tumor cell to a distant site, the method may comprise administering to a patient in need thereof, a compound or pharmaceutical composition as defined herein.

The present invention in yet a further aspect provides a method for reducing the growth of a metastatic cancer cell, the method may comprise contacting the metastatic cell with a compound or pharmaceutical composition as defined herein.

The present invention relates in another aspect to the use of a compound as defined herein, for reducing the growth of a metastatic cancer cell. In accordance with the present invention, the metastatic cancer cell may be, for example, a metastatic bone cancer cell arising from any type of metastatic cancer (tumor), such as, for example, a metastatic prostate cancer, a metastatic breast cancer, a metastatic lung cancer, a metastatic hepatocellular cancer or a metastatic myeloma or any type of cancer (tumor) able to metastasize to a bone.

The present invention in yet another aspect provides a pharmaceutical composition for inhibiting the growth of a metastatic cancer cell, the pharmaceutical composition may comprise a compound as defined herein and a pharmaceutically acceptable carrier.

The present invention further provides a method for reducing the expression of PTHrP in a cancer cell, the method may comprise contacting the cancer cell with a compound as defined herein. In accordance with the present invention, the cancer cell may be selected from the group consisting of a prostate cancer cell, a breast cancer cell, a lung cancer cell, a liver cell, a bone marrow cell or any other type of cancer cell having a high level of PTHrP. A high level PTHrP is to be understood herein as a value higher than normal. Normal values may be determined, for example, from values normally observed for a specific (healthy) individual or from the values normally observed in a (healthy) population.

The present invention in an additional aspect provides, a pharmaceutical composition for use in the reduction of the development of metastasis, the pharmaceutical composition may comprise a compound as defined herein (e.g., SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof) and a pharmaceutically acceptable carrier.

The present invention in yet an addition aspect, provides a method for reducing the development of metastasis, the method may comprise providing to a metastatic cancer cell, a compound as defined herein (e.g., SEQ ID NO.:1, SEQ ID NO.:2, SEQ ID NO.: 3, SEQ ID NO.: 4, SEQ ID NO.:5, SEQ ID NO.:6, biologicallyactive analogue (of anyone of SEQ ID NO.:1, SEQ ID NO.:2, SEQ ID NO.: 3, SEQ ID NO.: 4, SEQ ID NO.:5 (e.g., SEQ ID NO.:7), SEQ ID NO.:6) and combination thereof).

The present invention in a further aspect, relates to the use of a compound as defined herein in the manufacture of a pharmaceutical composition for reducing the development of a metastasis in a patient in need thereof.

It is to be understood that combinations of the compounds defined herein as well as combination of the compounds defined herein with other therapeutic coumpounds (e.g., anti-cancer drug, anti-metastatic cancer drug, anti-metastasis drug, drugs used for the treatment of hypercalcemia of malignancies, drug used to lower/inhibit the levels of expression, secretion, translation etc, of PTHrP or to reduce/block PTHrP-induced osteolysis , etc.) are also encompassed by the present invention. Therefore, the present invention relates to the use, methods, pharmaceutical compositions of the compounds defined herein (e.g., SEQ ID NO.:1, SEQ ID NO.:2, SEQ ID NO.: 3, SEQ ID NO.: 4, SEQ ID NO.:5, SEQ ID NO.:6, biologically active analogue (of any one of SEQ ID NO.:1, SEQ ID NO.:2, SEQ ID NO.: 3, SEQ ID NO.: 4, SEQ ID NO.:5 (e.g., SEQ ID. NO.:7), SEQ ID NO.:6) and combination thereof, etc.) in combination with hormone therapy, chemotherapy, radiation therapy or else. In accordance with the present invention, the compounds defined herein may be used with an antibody, an hormone or an anti-cancer drug, including for example, (without being restricted to) mitomycin, idarubicin, cisplatin, 5-fluoro-uracil, methotrexate, adriamycin, daunomycin, taxol (i.e., paclitaxel), and taxol derivative (e.g., docetaxel, taxane).

In accordance with the present invention, the metastasis may be, for example, a bone (skeletal) metastasis. According to the present invention, the condition may be selected from the group consisting of, for example, metastatic prostate cancer, metastatic breast cancer, metastatic lung cancer (e.g., lung carcinoma), metastatic hepatocellular cancer (e.g., hepatocellular carcinoma), metastatic myeloma or any other type of cancer which may have the potential of metastasizing to bone. The metastatic prostate cancer may be for example, a hormone refractory metastatic prostate cancer or a late stage hormone refractory metastatic prostate cancer. The compound may be, more particularly, SEQ ID NO.:5, a derivative, an analogue, a homologue (etc.) thereof. A SEQ ID NO.: 5 analogue may be for example, a SEQ ID NO.:5 biologically active analogue having an anti-metastatic effect. A SEQ ID NO.:5 analogue may be for example, a polypeptide set forth in SEQ ID NO.:7.

In accordance with the present invention, the metastatic cancer cell may be a metastatic bone cancer cell. The metastatic bone cancer cell may be, for example, from a cancer selected from the group consisting of a metastatic prostate cancer, a metastatic breast cancer, a metastatic lung cancer, a metastatic hepatocellular cancer and a metastatic myeloma, or from any other type of cancer able to metastasize to a bone. In accordance with the present invention, the metastatic cancer cell may be expressing PTHrP.

An analogue is to be understood herein as a compound (e.g. polypeptide) which retains at least partially, a biological activity of the original compound, i.e., a biologically active analogue. The (desired) biological activity of an analogue may be, for example an anti-tumor (growth) effect, an anti-metastasis effect (anti-metastatic effect, a reduction in metastatic potential, a reduced ability to promote tumor progression), an anti-invasive or anti-growth effect (or else) against a metastatic cancer, a modulation of a parathyroid hormone-related protein (PTHrP) level in a cell and/or in plasma of a patient, an anti-PTHrP-induced osteolysis effect, an effect against hypercalcemia of malignancy.

The biological activity of an analogue may be determined by contacting a tumor cell or a metastatic tumor cell (e.g., a cell expressing PTHrP) with a desired analogue and determining whether the analogue is a biologically active analogue by observing, for example, a reduction in cell growth. The biological activity of an analogue may also be determined, for example, in an in vivo model as described herein (i.e, Copenhagen rats injected with Mat Ly Lu cells (expressing or not PTHrP) where an effect against hypercalcemia of malignancy may be determined, for example, by a reduction in calcium levels upon injection of a biologically active analogue. The biological activity of an analogue may also be evaluated in a similar animal model where, following administration of an analogue, the levels of plasma PTHrP is measured or where the levels of PTHrP expression or production inside the cell is evaluated, as described herein, for example, using histologic methods. In such a case, a reduction of PTHrP levels (plasma levels or cell expression levels or both) is indicative of a biologically active analogue. An anti-metastatic effect of an analogue may be measured in an in vivo model as described herein, where a reduction in hind limb paralysis following injection of such analogue is indicative of a biologically active analogue. An anti-invasive effect of an analogue may be measured, for example, using 2-compartment Boyden Chamber (Transwell, Costar, Cambridge, Mass.) and basement membrane Matrigel assay as described herein. A decrease ability of a cell to invade in the presence of an analogue is indicative of a biologically active analog.

A biologically active analogue may have for example, a substitution of one or more amino acid, an addition of one or more amino acid, may have at least 50%, 70% or 90% of its amino acid sequence identical to that of PCK3145, may be modified or conjugated by the addition of a group (e.g., pegylation, RGD peptides) that may increase its bioavailability or modulate a desired characteristic of the compound, etc. Examples of PCK3145 biologically active analogue are given below.

As used herein, “polypeptides” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins. As described above, polypeptides may contain amino acids other than the 20 gene-encoded amino acids.

As used herein, the term “tumor” relates to solid or non-solid tumors, metastasic or non-metastasic tumors, tumors of different tissue origin including, but not limited to, tumors originating in the liver, lung, brain, lymph node, bone marrow, adrenal gland, breast, colon, pancreas, prostate, stomach, or reproductive tract (cervix, ovaries, endometrium etc.). The term “tumor” as used herein, refers also to all neoplastic cell growth and proliferation, whether malignant or benign, and all pre-cancerous and cancerous cells and tissues.

As used herein the term “metastasis” relates to a condition of a patient associated more particularly with the spread of tumor cells to distant organs. Therefore, it is to be understood herein that when compounds of the present invention are used to treat a condition such as metastasis (e.g., metastatic prostate cancer), they are used to reduce, lower, inhibit etc, one or more steps involved in the process of spreading of tumor cells to distant organs (e.g., tumor cell invasion).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions (medicaments) are liquids or lyophilized or otherwise dried formulations, granules and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally, etc.

Examples of excipients used for manufacturing solid pharmaceutical compositions include, for example, lactose, sucrose, starch, talc, cellulose, dextrin, kaolin, calcium carbonate and the like. Ordinarily used inert diluents such as vegetable oil may be added to liquid pharmaceutical compositions for oral administration, e.g., emulsions, syrups, suspensions, or solutions. These pharmaceutical compositions may also contain auxiliaries such as, for example, wetting agents, suspending aids, sweeteners, aromatic colorants, preservatives and the like in addition to the aforementioned inert diluents. Liquid pharmaceutical compositions may be encapsulated in capsules made of an absorbable material such as gelatin. Examples of a solvent or a suspending medium used for the preparation of pharmaceutical compositions for parenteral administration such as injections or drip infusions include, for example, water, propylene glycol, polyethylene glycol, benzyl alcohol, ethyl alcohol, ethyl oleate, lecithin or the like. The pharmaceutical formulations, compositions or medicament may be prepared, for example, by processes known in the art.

Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Mutant (variant, analogue, derivative) polypeptides encompassed by the present invention includes mutant that will possess one or more mutations, which are deletions (e.g., truncations), insertions (e.g., additions), or substitutions of amino acid residues. Mutants can be either naturally occurring (that is to say, purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques). Mutant polypeptide derived from PSP-94 (native PSP-94 (nPSP-94); SEQ ID NO.:1 or rHuPSP94 (recombinant human PSP-94): SEQ ID NO.:2) as well as derived from the polypeptide described herein (PCK3145 (SEQ ID NO.:5), decapeptide (SEQ ID NO.: 3), polypeptide 7-21 (SEQ ID NO.4), polypeptide 76-94 (SEQ ID NO.6)) having the biological activity described herein (effect on hypercalcemia, metastasis, etc) are included in the present application.

As may be appreciated, a number of modifications may be made to the polypeptides and fragments of the present invention without deleteriously affecting the biological activity of the polypeptides or fragments. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino or carboxy termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, pegylation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular proterties, 2^{nd} Ed., T. E. Creighton, W. H. Freeman and Company, New-York, 1993).

It is to be understood herein that, the peptide/compounds of the present invention may be conjugated with bisphosphonates, RGD peptides (Arginine-Glycine-Aspartic acid peptides), osteoblast, and osteoclast specific proteins to improve their bioavailability to the skeleton.

Other type of polypeptide modification may comprise, for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide. Polypeptides of the present invention comprise for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may have the biological property of polypeptides of the present invention which is to inhibit growth of prostatic adenocarcinoma, stomach cancer, breast cancer, endometrial, ovarian or other cancers of epithelial secretion, or benign prostate hyperplasia (BPH).

Example of substitutions may be those, which are conservative (i.e., wherein a residue is replaced by another of the same general type). As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same group as that of the amino acid be replaced. Thus, in some cases, the basic amino acids Lys, Arg and His may be interchangeable; the acidic amino acids Asp and Glu may be interchangeable; the neutral polar amino acids Ser, Thr, Cys, Gin, and Asn may be interchangeable; the non-polar aliphatic amino acids Gly, Ala, Val, lle, and Leu are interchangeable but because of size Gly and Ala are more closely related and Val, lle and Leu are more closely related to each other, and the aromatic amino acids Phe, Trp and Tyr may be interchangeable.

It should be further noted that if the polypeptides are made synthetically, substitutions by amino acids, which are not naturally encoded by DNA may also be made. For example, alternative residues include the omega amino acids of the formula NH₂(CH₂)ₙCOOH wherein n is 2-6. These are neutral nonpolar amino acids, as are sarcosine, t-butyl alanine, t-butyl glycine, N-methyl isoleucine, and norleucine. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that mutants or variants may be generated by substitutional mutagenesis and retain the biological activity of the polypeptides of the present invention. These variants have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (lle)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro); and
(6) aromatic: Tryptophan (Trp), Tyrosine (Tyr), Phenylalanine (Phe)

Non-conservative substitutions will entail exchanging a member of one of these classes for another.

TABLE 1
Preferred amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Example of biologically active analogues of PCK3145 (SEQ ID NO: 5) exemplified by amino acid substitutions is illustrated below.

| | |
|---|---|
| Position | 1 5 10 15 |
| PCK3145 | E W Q T D N C E T C T C Y E T |
| (SEQ ID | X₁ W Q X₂ D X₁ C X₁ X₂ C X₂ C X₃ X₁ X₂ |
| NO.:7) | |

For example, X₁ may be glutamic acid (i.e., glutamate) (Glu), aspartic acid (aspartate) (Asp), or asparagine (Asn), X₂ may be threonine (Thr) or serine (Ser) and X₃ may be tyrosine (Tyr) or phenylalanine (Phe).

Polypeptides that are polypeptide analogues of PSP-94 (nPSP-94 (SEQ ID NO.:1) or rHuPSP94 (SEQ ID NO.:2)) and/or analogues of PCK3145 (SEQ ID NO.:5), the decapeptide (SEQ ID NO.: 3), the polypeptide 7-21 (SEQ ID NO.4), the polypeptide 76-94 (SEQ ID NO.6)) include, for example, the following:
a polypeptide analogue of at least five contiguous amino acids of SEQ ID NO: 2, of SEQ ID NO: 3, of SEQ ID NO: 4, of SEQ ID NO: 5, or of SEQ ID NO: 6;
a polypeptide analogue of at least two contiguous amino acids of SEQ ID NO: 2, of SEQ ID NO: 3, of SEQ ID NO: 4, of SEQ ID NO: 5, or of SEQ ID NO: 6;
a polypeptide analogue consisting of the amino acid sequence X₁ W Q X₂D X₁C X₁X₂C X₂C X₃X₁X₂ (SEQ ID NO.:7), wherein X₃ is either glutamic acid (Glu), asparagine (Asn) or aspartic acid (Asp), X₂ is either threonine (Thr) or serine (Ser), and X₃ is either tyrosine (Tyr) or phenylalanine (Phe);
a polypeptide analogue comprising SEQ ID NO: 5 and having an addition of at least one amino acid to its amino-terminus;
a polypeptide analogue comprising SEQ ID NO: 5 and having an addition of at least one amino acid to its carboxy-terminus;
a polypeptide analogue comprising two to ten units of SEQ ID NO: 5;
a polypeptide analogue comprising two to fifty units of SEQ ID NO: 5;
a polypeptide analogue consisting of a sequence of from two to fourteen amino acid units wherein the amino acid units are selected from the group of amino acid units of SEQ ID NO: 5 consisting of glutamic acid (Glu), tryptophan (Trp), glutamine (Gln), threonine (Thr), aspartic acid (Asp), asparagine (Asn), cysteine (Cys), or tyrosine (Tyr);
a polypeptide analogue having at least 90% of its amino acid sequence identical to the amino acid sequence set forth in SEQ ID NO: 5;
a polypeptide analogue having at least 70% of its amino acid sequence identical to the amino acid sequence set forth in SEQ ID NO: 5;
and a polypeptide analogue having at least 50% of its amino acid sequence identical to the amino acid sequence set forth in SEQ ID NO: 5;
or any polypeptide analogue of PSP-94 (nPSP-94 (SEQ ID NO.:1) or rHuPSP94 (SEQ ID NO.:2)) as well as the derivative described herein (PCK3145 (SEQ ID NO.:5), decapeptide (SEQ ID NO.: 3), polypeptide 7-21 (SEQ ID NO.4), polypeptide 76-94 (SEQ ID NO.6)) having the biological activity described herein (effect on hypercalcemia, bone metastasis, etc.).
Examples of a derivative where homologous sequences are fused to PCK3145 (SEQ ID NO: 5) are also illustrated in Canadian patent application No: 2,359,650 which is incorporated herein by reference.

Amino acids sequence insertions (e.g., additions) include amino and/or carboxyl-terminal fusions ranging in length from one residues to polypeptides containing a hundred or more residues, as well as intrasequence insertions of single or multiple amino acid residues. Other insertional variants include the fusion of the N- or C-terminus of the protein to a homologous or heterologous polypeptide forming a chimera. Chimeric polypeptides (i.e., chimeras, polypeptide analog) comprise sequence of the polypeptides of the present invention fused to homologous or heterologous sequence. The homologous or heterologous sequence encompass those which, when formed into a chimera with the polypeptides of the present invention retain one or more biological or immunological properties.

A protein which is at least 50% identical, as determined by methods known to those skilled in the art (for example, the methods described by Smith, T. F. and Waterman M. S. (1981) Ad. Appl.Math., 2:482-489, or Needleman, S. B. and Wunsch, C. D. (1970) J.Mol.Biol., 48: 443-453), to those polypeptides of the present invention are included in the invention, as are proteins at least 70% or 80% and more preferably at least 90% identical to the protein of the present invention. This will generally be over a region of at least 5, preferably at least 20 contiguous amino acids.

Examples of PSP-94 analogues may be found, for example, in the protein sequence database of the National Center for Biotechnology Information web site (www.ncbi.nlm.nih.gov/) under accession numbers, 1209281A, Q28767, AAB62726.1, P25142, 097936, 097949, 097935, CAB39325.1, AAB50711.1, S41663, AAB19102.1, etc.

It is to be understood herein that the methods, reagents or else exemplified herein (cell culture conditions, cell type, animal protocols, etc.) are only given by way of example and may be performed as successfully by other methods, reagents or else known in the art.

It is to be understood herein, that if a “range” or “group” of substances (e.g. amino acids), “substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,
with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C. up to 210° C., etc.;
with respect to reaction time, a time of 1 minute or more is to be understood as specifically incorporating herein each and every individual time, as well as sub-range, above 1 minute, such as for example 1 minute, 3 to 15 minutes, 1 minute to 20 hours, 1 to 3 hours, 16 hours, 3 hours to 20 hours etc.;
with respect to polypeptides, a polypeptide analogue consisting of at least two contiguous amino acids of a particular sequence is to be understood as specifically incorporating each and every individual possibility, such as for example, a polypeptide analogue consisting of amino acid 1 and 2, a polypeptide analogue consisting of amino acids 2 and 3, a polypeptide analogue consisting of amino acids 3 and 4, a polypeptide analogue consisting of amino acids 6 and 7, a polypeptide analogue consisting of amino acids 9 and 10, a polypeptide analogue consisting of amino acids 36 and 37, a polypeptide analogue consisting of amino acids 93 and 94, etc.
and similarly with respect to other parameters such as, concentrations, elements, etc . . .

It is in particular to be understood herein that the polypeptides of the present invention each include each and every individual polypeptide described thereby as well as each and every possible mutant, variant, homolog, analogue or else whether such mutant, variant, homolog, analogue or else is defined as positively including particular polypeptides, as excluding particular polypeptides or a combination thereof; for example an exclusionary definition for a polypeptide analogue (e.g. X₁WQX₂DX₁CX₁X₂CX₂CX₃X₁X₂ (SEQ ID NO.:7)) may read as follows: “provided that when one of X₁ is glutamic acid and X₂ is threonine X₃ may not be phenylalanine”.

It is also to be understood herein that “g” or “gm” is a reference to the gram weight unit; that “C” is a reference to the Celsius temperature unit.

### BRIEF DESCRIPTION OF THE DRAWINGS

In drawings which illustrate exemplary embodiments of the present invention:

FIG. 1 is a graph illustrating the effect of PSP-94 on Mat Ly Lu-PTHrP cell growth. Each point represents the mean of 3 different experiments. Significant differences from control cells (MatLyLu-CMV) and PTHrP transfected cells (MatLyLu-PTHrP) in the abscence of PSP-94 are represented by asterisks (p<0.05);

FIG. 2A is a histogram illustrating the effect of PSP-94 on Mat Ly Lu-PTHrP tumor volume (in cm³). Results represent the mean ± SEM of 5 animals in each group in 3 different experiments. Significant difference from control tumor-bearing animals receiving vehicle alone (CTL) are represented by asterisks (p<0.05);

FIG. 2B is also a histogram illustrating the effect of PSP-94 on Mat Ly Lu-PTHrP tumor volume (in cm³). Results represent ± SEM of six animals in each group. Significant difference in tumor volume is shown by asterisks (p<0.05);

FIG. 3 is a graph illustrating the effect of PSP-94 on animal weight. Results represent the mean ± SEM of 5 animals in each group in 3 different experiments;

FIG. 4 is a histogram illustrating the effect of PSP-94 on Mat Ly Lu-PTHrP tumor weight. Results represent the mean ± SEM of 5 animals in each group in 3 different experiments. Significant difference from control tumor-bearing animals receiving vehicle alone (CTL) are represented by asterisks (p<0.05);

FIG. 5A is a graph illustrating the effect of PSP-94 on spinal metastasis resulting in the development of hind limb paralysis. Results represent the mean ± SEM of 5 animals in each group in 3 different experiments. Significant difference in % of non-paralyzed animals from control tumor-bearing animals receiving vehicle alone (CTL) are represented by asterisks (p<0.05);

FIG. 5B is also a graph illustrating the effect of PSP-94 on spinal metastasis resulting in the development of hind limb paralysis;

FIG. 6 is a histogram illustrating the effect of PCK-3145 on experimental skeletal metastasis resulting in the development of hind limb paralysis. Results represent the mean ± SEM of 5 animals in each group in 3 different experiments. Significant differences in percentage of non-paralyzed animals from control tumor-bearing animals receiving vehicle alone (CTL) are represented by asterisks (p<0.05);

FIG. 7A are photographs of histological sections illustrating the effect of PCK3145 treatment on the degree of skeletal metastasis compared to control (CTL) animals. A representative photomicrograph of three experiments is shown. Magnification 200×;

FIG. 7B is a histogram illustrating the effect of PCK3145 treatment on the degree of skeletal metastasis compared to control (CTL) animals and expressed as the ratio of tumor volume to bone volume (TV/BV). Results represent the mean ± SEM of 5 animals in each group in 3 different experiments. Significant differences in the ratio of tumor volume to bone volume from control tumor-bearing animals receiving vehicle alone are represented by asterisks (p<0.05);

FIG. 8A are photographs of histological sections illustrating the effect of PCK3145 treatment on PTHrP production in experimental skeletal metastasis compared to control animals (CTL). Representative photomicrograph of three experiments is shown. Magnification 200×, and;

FIG. 8B is a histogram illustrating the effect of PCK3145 treatment on PTHrP production in experimental skeletal metastasis compared to control animals (CTL). Levels of PTHrP production in control and experimental metastasis were quantified and expressed as total density. Results represent the mean ± SEM of 5 animals in each group in 3 different experiments. Significant differences in the levels of PTHrP production from control tumor-bearing animals receiving vehicle alone are represented by asterisks (p<0.05).

FIG. 9A is a histogram illustrating the effect of PSP-94 on plasma PTHrP in tumor bearing animals. Results represent the mean ± SEM of 5 animals in each group in 3 different experiments. Significant difference from control tumor-bearing animals receiving vehicle alone (CTL) are represented by asterisks (p<0.05). Results obtained for non-tumor bearing animals (N) are also illustrated;

FIG. 9B is a histogram illustrating the effect of PSP-94 on plasma calcium in tumor bearing animals. Results represent the mean ± SEM of 5 animals in each group in 3 different experiments. Significant difference from control tumor-bearing animals receiving vehicle alone (CTL) are represented by asterisks (p<0.05). Results obtained for non-tumor bearing animals (N) are also illustrated;

FIG. 10A is also a histogram illustrating the effect of PSP-94 on plasma PTHrP in tumor bearing animals. Results represent ± SEM of 6 different animals in each group. Significant difference from control (CTL) is marked by asterisks (p<0.05). Results obtained for non-tumor bearing animals (N) are also illustrated;

FIG. 10B is a further histogram illustrating the effect of PSP-94 on plasma calcium of tumor bearing animals. Results represent ± SEM of 6 different animals in each group. Significant difference from control (CTL) is marked by asterisks (p<0.05). Results obtained for non-tumor bearing animals (N) are also illustrated; FIG. 11 are photographs of histologic sections illustrating the effect of different doses of PSP-94 on PTHrP production by Mat Ly Lu-PTHrP tumors compared to control (CTL). A representative photomicrograph of three experiments is shown at a magnification 200×;

FIG. 12A is a histogram illustrating the effect of PCK-3145 on plasma PTHrP levels in tumor bearing animals using a radioimmunoassay. Results represent the mean ± SEM of 5 animals in each group in 3 different experiments. Significant differences from control tumor-bearing animals receiving vehicle alone (CTL) are represented by asterisks (p<0.05). Results obtained for non-tumor bearing animals (N) are also illustrated;

FIG. 12B is a histogram illustrating the effect of PCK-3145 on plasma calcium levels in tumor bearing animals. Results represent the mean ± SEM of 5 animals in each group in 3 different experiments. Significant differences from control tumor bearing animals receiving vehicle alone (CTL) are represented by asterisks (p<0.05). Results obtained for non-tumor bearing animals (N) are also illustrated;

FIG. 13 are photographs of histologic sections illustrating the effect of PCK3145 on PTHrP production by Mat Ly Lu-PTHrP tumors compared to controls (CTL, NC). A representative photomicrograph of three experiments is shown. Magnification 200×. Results are representative of three animals in each group and three tumor sections which were analyzed from each animal by evaluating at least ten random fields of observation;

FIG. 14A is a photograph of an agarose gel illustrating the effect of PSP-94 on DNA fragmentation of Mat Ly Lu-PTHrP cells in vitro compared to control treated cells (CTL). A representative photograph of three experiments is shown;

FIG. 14B are photographs illustrating the effect of PSP-94 on DNA fragmentation of Mat Ly Lu-PTHrP cells in vivo compared to control treated cells (CTL). All animals were sacrificed at the end of the study and their primary tumors removed, paraffin embedded, sectioned and processed by TUNEL assay as described herein (upper panel) or counterstained with Hoescht reagent (lower panel). Three animals were present in each group and three sections were analyzed for each animal. At least ten random fields of observation were evaluated. A representative photomicrograph for three such experiments in each group is shown. Magnification 200×;

FIG. 15 is a graph illustrating the effect of PCK3145 on Mat Ly Lu-PTHrP cell growth compared to control (CTL). Results represent the mean of 3 different experiments where significant differences from control cells is represented by asterisks (p<0.05);

FIG. 16 is a histogram illustrating the effect of PCK3145 on Mat Ly Lu-PTHrP tumor volume. Results shown in panel A represent the mean ± SEM of 5 animals in each group in 3 different experiments. Significant differences from control tumor-bearing animals receiving vehicle alone are represented by asterisks (p<0.05);

FIG. 17 are photographs illustrating the effect of PCK3145 on DNA fragmentation of Mat Ly Lu-PTHrP cells in vivo compared to control treated cells (CTL). Five animals were present in each group and three sections were analyzed for each animal. At least ten random fields of observation were evaluated. A representative photomicrograph for three experiments in each group is shown. Sections were processed by TUNEL assay (upper panel) and counterstained with Hoescht reagent (lower panel). Magnification 200×;

### DETAILED DESCRIPTION OF THE INVENTION

In the present study, the capacity of PSP-94 and PCK3145 to counteract the ability of tumor cells to invade distant sites and to specifically metastasize to bone tissue was evaluated. Furthermore, the effect of PSP-94 (e.g., SEQ ID NO.:1, SEQ ID NO.:2) and PCK3145 (SEQ ID NO.:5) on metastatic cancer cell (e.g., skeletal metastasis, metastatic prostate cancer) was studied herein.

PSP-94 (native) (SEQ ID NO.:1) may be generated, for example, as described in U.S. Pat. No.: 5,428,011. Recombinant PSP-94 (rHuPSP-94; SEQ ID NO.:2) and PCK3145 (SEQ ID NO.:5) may be generated as described for example, in Canadian patent application No.: 2,359,650 filed on Oct. 15, 2001.

For this purpose, MatLyLu rat prostate cancer cells were transfected with full-length cDNA encoding parathyroid hormone related protein (PTHrP). MatLyLu-PTHrP cells were inoculated subcutaneously (S.C.) into the right flank or via intracardiac route (I.C.) into the left ventricle of syngenic male Copenhagen rats. Intracardiac inoculation of MatLyLu-PTHrP cells allows these cells to directly enter the blood stream and routinely results in tumor metastasis to bone, the preferential target site of these cells. More particularly, metastases to bone are found to the lumbar vertebrae which result in hind limb paralysis. Time of hind limb paralysis and tumor volume was measured and comparison was made between PSP-94- or PCK3145-treated animals and control animals receiving vehicle alone. At the end of the study, animals were sacrificed and serum Ca⁺² (calcium, Ca⁺⁺) and PTHrP levels in control and experimental animals were determined. Primary tumors and skeletal metastasis to lumbar vertebrae were also examined for PTHrP production by immunohistochemistry. Affected lumbar vertebras were also removed for radiological and histological analysis. Evidence of tumor cell apoptosis was monitored by subjecting histological specimens to Hoechst staining and TUNEL assays.

Animal Protocols. Inbred male Copenhagen rats weighing 200-250g were obtained from Harlan Sprague-Dawley (Indianapolis, Ind.). Before inoculation, Mat Ly Lu-PTHrP tumor cells growing in serum-containing medium were washed with Hanks buffer, trypsinized, and collected by centrifugation at 1500 rpm for 5 min. (Achbarou, A. et al., Cancer Res., 54:2372-2377,1994; Rabbani, S. A. et al., Int. J. Cancer, 80: 257-264, 1999; Rabbani, S. A. et al., Endocrinology, 136:5416-5422, 1995). Cell pellets (10×10³ cells) were resuspended in 100 ul saline and injected using 1 ml syringes into the left ventricle of rats anaesthetised with ketamine/xylazine cocktail. Animals were divided into control groups which received vehicle alone (PBS: phosphate buffered saline) and experimental groups which were infused I.P. with different doses (0.1-10.0 ug/kg/day) of PSP-94 starting at the time of tumor cell inoculation (day 0) until the day of skeletal metastasis development. The time after tumor cell inoculation which was required to develop hind limb paralysis (an index of spinal cord compression due to lumbar vertebrae metastasis) was determined and percentage of starting number of animals developing hind-limb paralysis was plotted.

Alternatively, cell pellets (10×10³ cells) were resuspended in 100ul saline and injected using 1 ml syringes into the left ventricle of rats anaesthetised with ketamine/xylazine cocktail. Animals were divided into control groups which received vehicle alone (PBS) and experimental groups which were infused via intraperitoneal injection (I.P.) with different doses (1.0-100.0 ug/kg/day) of PCK 3145 starting at the time of tumor cell inoculation (day 0) until the day of skeletal metastasis development. The time after tumor cell inoculation which was required to develop hind limb paralysis (an index of spinal cord compression due to lumbar vertebrae metastasis) was determined and percentage of starting number of animals developing hind-limb paralysis was plotted. In other sets of experiments, following I.C. tumor cell inoculation, control animals receiving PBS and experimental animals receiving 100.0 ug/kg/day of PCK3145 were sacrificed before the development of hind-limb paralysis (day 10 post tumor cell inoculation) and their vertebral column spanning from L1-L5 was removed and subjected to bone histomorphometric and immunohistochemical analysis as described herein.

Also, alternatively, cell pellets (5×10⁵ cells) were resuspended in 100 ul saline and injected using 1 ml syringes into the right flank of rats as described herein. From the time of tumor cell inoculation, experimental animals were treated with different doses (0.1, 1.0, 10.0, or 100.0 ug/kg/day) of PSP-94 or PCK3145 (SEQ ID NO.:5) via S.C. injections for 15 consecutive days. Control animals received PBS alone as vehicle control. All animals were numbered, kept separately and monitored daily for the development of tumors. The tumor mass was measured in 2 dimensions by calipers and tumor volume was calculated according to the equation (/x w²)/2 (/=length, w=width) (Rabbani, S. A. et al., Int. J. Cancer, 80: 257-264, 1999; Rabbani, S. A. et al., Endocrinology, 136:5416-5422,1995). All control and experimental animals were weighed every alternate day to determine any adverse effect of PSP-94 or PCK3145. Both control and experimental animals were sacrificed at day 16 post tumor cell inoculation and their tumors were removed and weighed. Additionally, these tumors were used for histological analysis as described herein. Blood from all control and experimental animals was collected on day 16 for determination of plasma Ca²⁺ and PTHrP levels.

Cells and cell culture. The Dunning R3327 Mat Ly Lu cell line (available, for example, under American Type Culture Collection No.: JHU-5) was transfected with full length cDNA encoding rat PTHrP as previously described (Rabbani, S. A. et al., Int. J. Cancer, 80:257-264,1999). One of the three well characterized monoclonal cell lines Mat Ly Lu-PTHrP-8 was used throughout the course of the present studies. Cells were maintained in vitro in RPMI 1640 supplemented with 2 mM L-glutamine (Life Technologies, Inc. Grand Island, N.Y.), 10% foetal bovine serum (FBS), 100 units/ml penicillin-streptomycin sulphate (Life Technologies, Inc.), and 250 nM dexamethasone and G418 (600 mg/ml) according to previously established methods of culture of these experimental cells (Rabbani, S. A. et al., Int. J. Cancer, 80: 257-264, 1999).

Cell morphology. Morphological analysis of control and experimental Mat Ly Lu-PTHrP cells treated with PSP-94 or PCK3145 was carried out by plating 5×10⁴ cells/ well in 6-well plates (Falcon Plastics, Oxnard, Calif.) in the presence of 10% FBS. Cells were examined daily for any change in their morphology and photographed (Rabbani, S. A. et al., Endocrinology, 136:5416-5422,1995).

Invasion. Effect of PSP-94 on Mat Ly Lu-PTHrP tumor cell invasive capacity is examined by 2-compartment Boyden Chamber (Transwell, Costar, Cambridge, Mass.) and basement membrane Matrigel (Becton Dickinson Labware, Bedford Mass.) as previously described (Liu, D. F. et al., Prostate, 27:269-276,1995).

Growth curve. For growth curves, Mat Ly Lu-PTHrP cells were plated in 6-well plates (Falcon Plastics, Oxnard, Calif.) at seeding densities of 5×10³ cells/well. Mat Ly Lu-PTHrP cells were grown in the presence of 0.1, 1.0 & 10.0 ug/ml of PSP-94 or vehicle alone for up to 3 days and the ability of PSP-94 to alter cell doubling time was evaluated daily. Medium was changed every two days. The number of cells was counted in a model Z Coulter counter (Coulter Electronics, Beds, UK). Comparison was also made with doubling time of wild type untransfected Mat Ly Lu cells.

Histologic Analysis. For immunohistological analysis, primary tumor samples were dewaxed by heating at 60° C. and rehydrated in a graded alcohol series (100%-70%). Anti-rat antibody against PTHrP was used as the primary antibody. Tumor sections were incubated overnight at 4° C. followed by further incubation with biotinylated universal antibody (Vector Laboratories, Burlingame, Calif.) for 45-60 minutes. Sections were rinsed with TBST (Tris buffered saline-Tween) followed by incubation with Vectastain ABC-AP Reagent (Vector Laboratories, Burlingame, Calif.) for 30 minutes. These sections were again washed with TBST and incubated with a Napthol AS-Mix Phosphate/Fast Red solution (Sigma-Aldriche, Oakville, ON). The sections were finally counterstained with Methyl Green (Vector Laboratories, Burlingame, Calif.) and mounted.

Alternatively, paraffin embedded tumor samples were cut into 5 um-thick sections for immunohistochemical analysis. Immunohistochemical staining for PTHrP was performed as described in Pizzi, H. et al. (Endocrinology, 144:858-867, 2003) using the avidin-biotin-peroxidase complex method (Hsu, S. M. et al, J. Histochem Cytochem., 29:577-580, 1981). Briefly, the sections were dewaxed in xylene, and rehydrated through a series of ethanol to water gradients. The sections were incubated in 1% normal goat sera (Vector Laboratories Inc., Burlingame, Calif., USA) for 30 min at room temperature before treatment with the primary antibody (polyclonal antiserum against PTHrP (1-34) from rabbit) at 1:200 dilution overnight at 4° C. Biotinylated goat anti-rabbit IgG (Vector Laboratories Inc., Burlingame, Calif., USA) was used as the secondary antibody at a dilution of 1:200 for 30 min at room temperature. The slides were treated with Vectastain ABC-AP kit (Vector Laboratories Inc., Burlingame, Calif., USA) diluted 1:200 for 30 min at room temperature, and subsequently developed with Fast Red TR/Naphthol AS-MX phosphate (Sigma-Aldrich Canada) containing 1 mM levamisole for 10-15 minutes. The slides were then counterstained with hematoxylin (Fisher Scientific Ltd, Nepean, ON, Canada) and mounted with Kaiser's glycerol jelly. All sections were washed three times; ten minutes each, with Tris buffer (pH 7.6) after each step. For negative control sections, the primary antibody was omitted.

For example, vertebral columns were fixed and decalcified for a period of 3 weeks. Vertebral column samples were dewaxed in xylene, and rehydrated through a graded alcohol series (100%-70%). For determining the level of expression of PTHrP by skeletal metastasis, the sections were incubated in 1% normal goat serum (Vector Laboratories Inc., Burlingame, Calif., USA) for 30 min at room temperature before treatment with the primary antibody (polyclonal antiserum against PTHrP (1-34) from rabbit) at 1: 200 dilution overnight at 4° C. Biotinylated goat anti-rabbit IgG (Vector Laboratories Inc., Burlingame, Calif., USA) was used as the secondary antibody at 1:200 for 30 min at room temperature. The slides were treated with Vectastain ABC-AP kit (Vector Laboratories Inc., Burlingame, Calif., USA) diluted 1:200 for 30 min at room temperature, and subsequently developed with Fast Red TR/Naphthol AS-MX phosphate (Sigma-Aldrich, Oakville, QN, Canada) containing 1 mM levamisole for 10-15 minutes. The slides were then counterstained with hematoxylin (Fisher Scientific Ltd, Nepean, ON, Canada) and mounted with Kaiser's glycerol jelly. All sections were washed three times; ten minutes each, with Tris buffer (pH 7.6) after each step.

For example, for bone histomorphometry analysis, decalcified vertebrae from control and experimental animals were fixed and embedded in paraffin. 5 um sections of the decalcified vertebrae sections were then used for analysis and stained with Hematoxylin and Eosin (H&E staining). The H&E stained vertebral sections were then used to determine percentage of tumor volume/bone volume as described herein.

For TUNEL assay, tissue sections were, for example, dewaxed (e.g., in xylene), (may also be rehydrated by heating at 60° C. followed by washing in xylene) and (further) rehydrated through a graded alcohol series (100%-70%). Tissues were incubated with proteinase K for 30 min at 37° C. fixed, blocked and permeabilized. Apoptotic cells were detected by TUNEL assay in situ cell death detection kit (Roche Molecular Biochemicals, Laval, QC) according to the manufacturer's instruction. Positive TUNEL staining was visualised by fluorescence microscopy. In other experiments following TUNEL assay, tissue sections were counterstained with Hoechst 33258 (Sigma-Aldrich, Canada). Hoechst staining was added to tissues at a final concentration of 24 ug/ml in PBS and incubated for 15 minutes at room temperature. Tissue sections were washed and visualized by fluorescence microscopy using a blue screen (Rabbani, S. A., et al., Int. J.Cancer, 87:276-282, 2000). All results of immunohistochemistry and TUNEL assay were evaluated and interpreted by two independent examiners.

Computer-Assisted Image Analysis. Computer-assisted image analysis was carried out to quantify PTHrP immunostaining and determination of % TV/BV in the vertebral sections. Briefly, images of stained sections were photographed with a Leica digital camera and processed using BioQuant image analysis software, version 6.50.10 (BioQuant Image Analysis Corporation, Nashville, Tenn., USA). The threshold was set by determining the positive staining of control sections and was used to automatically analyze all recorded images of all samples that were stained in the same session under identical conditions. The area of stained regions was calculated automatically by the software in each microscopic field. Pixel counts of the immunoreaction product were calculated automatically and were given as total density of the integrated immunostaining over a given area.

Other Analytical Methods. Plasma calcium levels were determined by atomic absorption spectrophotometry (model 703, Perkin-Elmer, Norwalk, Conn.). For plasma PTHrP, all samples were tested in two dilutions in PTHrP radio-immunoassay (R.I.A.) kit (Nichols Institute Diagnostics, San Juan Capistrano, Calif.) according to manufacturer's instructions.

Statistical Analysis. Results are expressed as the mean ± SEM (standard error) of at least triplicate determinations, and statistical comparisons are based on the Student's t test or analysis of variance (ANOVA). A probability value of <0.05 was considered to be significant (Glantz, S. A., Primer of biostatistics, McGraw-Hill, N.Y., 1981). Regression analysis was used to determine the effect of PCK3145 on Mat Ly Lu-PTHrP cell growth.

### EXAMPLE 1

### Effect of PSP-94 on MatLyLu-PTHrP Cell Growth, Morphology and Invasion.

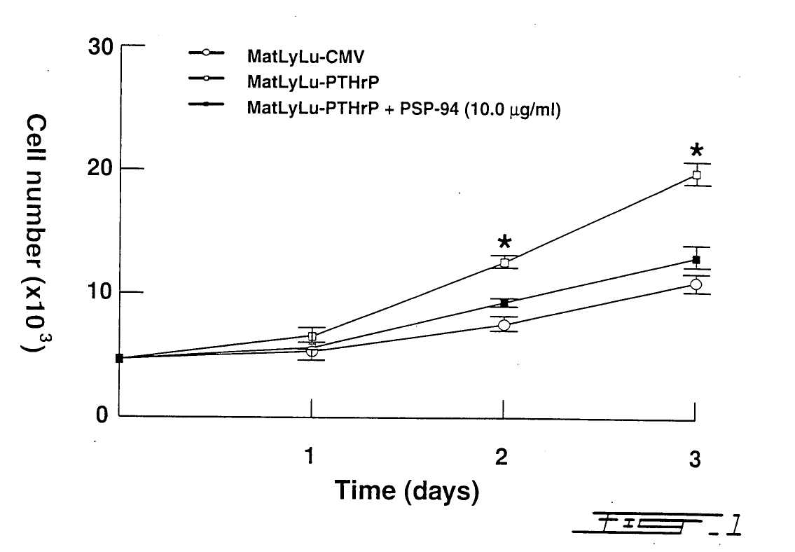

Mat Ly Lu cells transfected with vector alone (CMV) or vector expressing PTHrP were seeded at a density of 5×10³ cells/well in 6-well plates. Mat Ly Lu-PTHrP cells were treated with PSP-94 and were trypsinized and counted using a coulter counter as described herein. Change in cell number following treatment with 10.0 ug/ml of PSP-94 for 72 hrs is illustrated in FIG. 1. Transfection of Mat Ly Lu with PTHrP cDNA resulted in reduced doubling time and increase in tumor cell growth due to the growth promoting effects of PTHrP. Thus, Mat Ly Lu-PTHrP cells had a higher rate of cell proliferation as compared to control Mat Ly Lu cells transfected with vector a lone. A significant decrease in MatLyLu-PTHrP cell growth was seen following treatment with 10.0 ug/ml of PSP-94 for 72 hrs (FIG. 1). Treatment of Mat Ly Lu-PTHrP cells with 10.0 ug/ml of PSP-94 for 3 days resulted in a noticeable change in tumor cell morphology where tumor cells were found to change their normal spindle-like shape to a more rounded and condensed appearance (data not shown).

### EXAMPLE 2

### Effect of PSP-94 on Mat Ly Lu-PTHrP Tumor Growth in vivo.

Male Copenhagen rats were inoculated with Mat Ly Lu-PTHrP cells (1×10⁶ cells) via S.C. route of injection into the right flank as described herein. Starting from the day of tumor cell inoculation animals were infused S.C., below the tumor cell inoculation site, with different doses of PSP-94 (0.1-10.0 ug/kg/day) for up to 15 days. Effect of PSP-94 on reducing tumor growth was evaluated by daily determination of tumor volume with comparison being made to control tumor-bearing animals receiving vehicle alone.

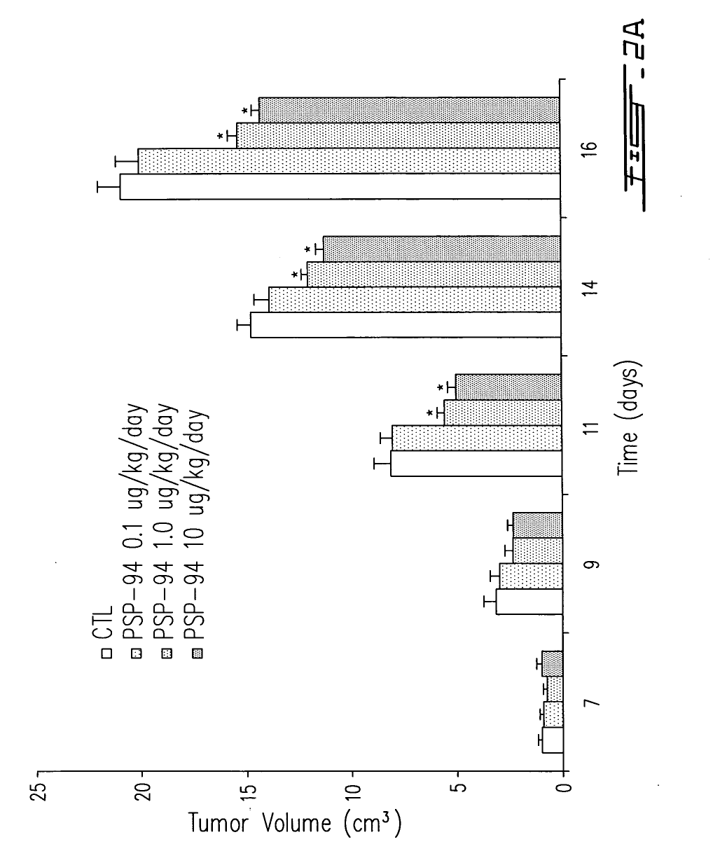

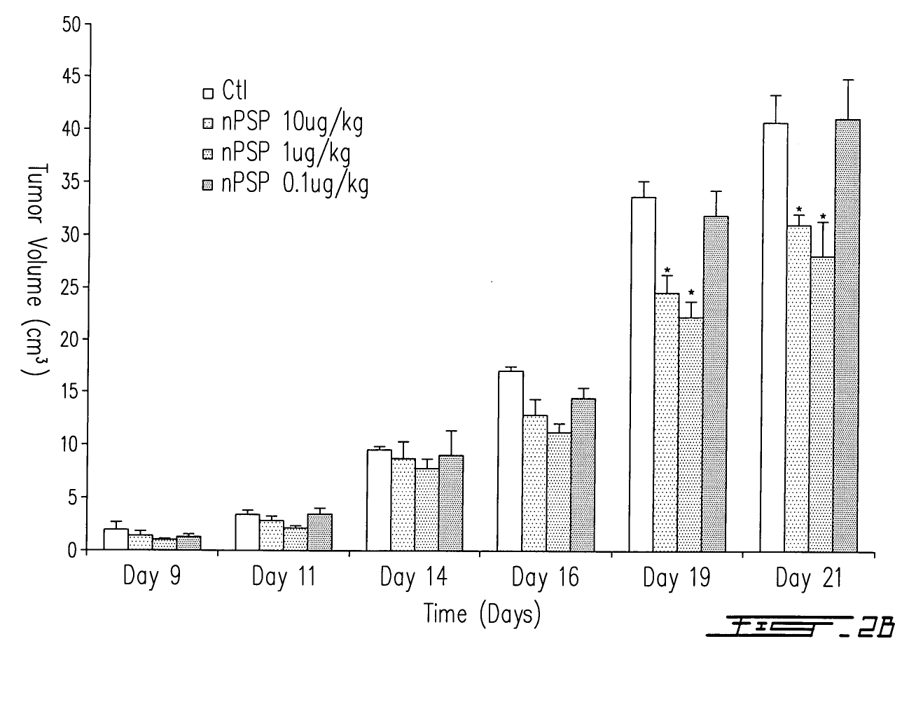

Tumor volume was measured at timed intervals and comparison was made with that of tumor-bearing animals receiving vehicle alone as control (CTL). In FIG. 2B male Copenhagen rats were inoculated s.c with 10⁶ Mat Ly Lu-PTHrP cells. After 3 days of tumor cell inoculation, animals were injected with vehicle alone (Ctl) or different doses (0.1, 1, 10 ug/kg) of PSP-94 (nPSP) at the site of tumor cell injection. Tumor volume (expressed in cubic centimeter (cm³)) was determined at timed intervals. Results presented in FIG. 2A and FIG. 2B indicate that control animals showed a progressive increase in tumor volume throughout the duration of the study. In contrast to this, experimental animals receiving PSP-94 showed a marked dose-dependent reduction in tumor volume throughout the course of this study (FIG. 2A, 2B).

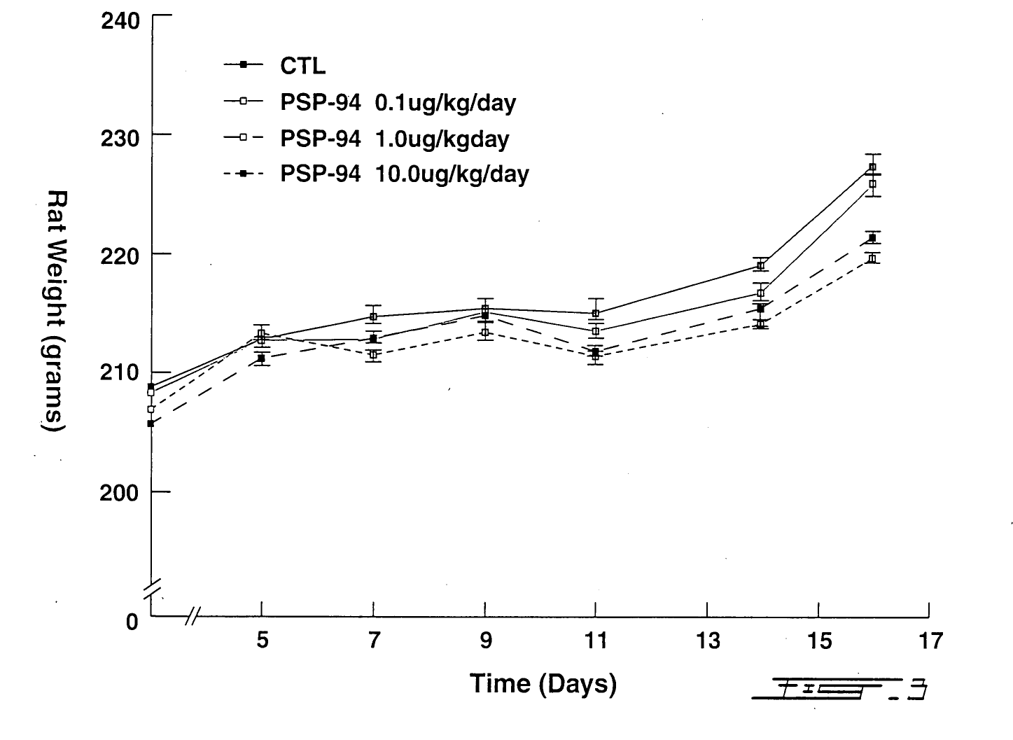

During the study, both control and experimental animals were monitored for any noticeable side effects and cachexia resulting in weight loss. These results presented in FIG. 3 were obtained from male Copenhagen rats injected S.C. into the right flank with 1×10⁶ Mat Ly Lu-PTHrP cells. Starting on the time of tumor cell inoculation animals were infused with different doses of PSP-94 for fifteen consecutive days as described herein. All animals were weighed at timed intervals and comparison was made with that of tumor bearing animals receiving vehicle alone as control (CTL). No significant change in the weight of control and experimental groups of animals that can be attributed to any potential side effect of PSP-94 treatment was observed (FIG. 3).

### EXAMPLE 3

### Effect of PSP-94 on Mat Ly Lu-PTHrP Tumor Weight.

In order to determine the effect of PSP-94 on tumor weight, animals inoculated with Mat Ly Lu-PTHrP via S.C. route of injection were sacrificed at the end of the study (day 16) and their tumors excised and weighed.

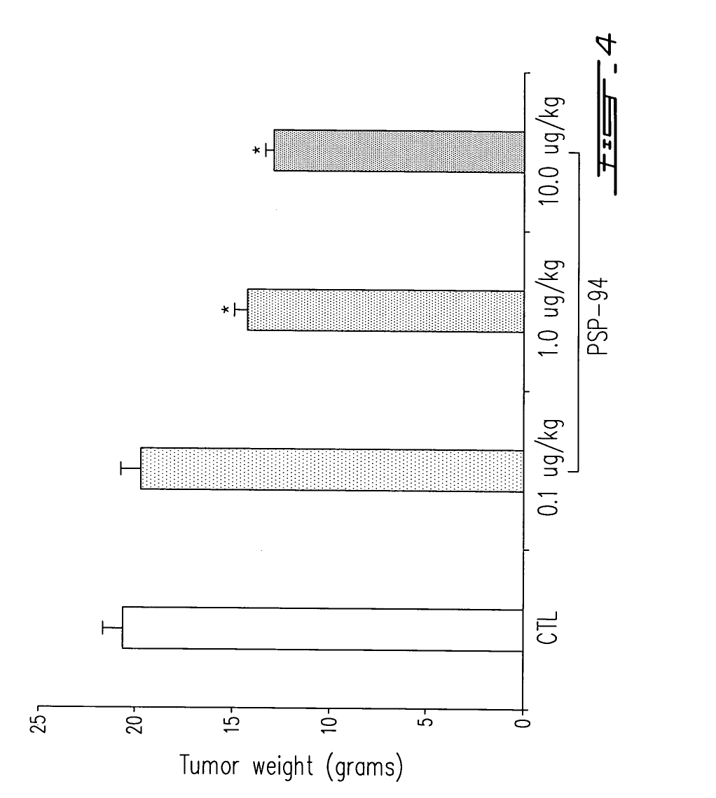

Results presented in FIG. 4 shows Male Copenhagen rats inoculated with 1×10⁶ Mat Ly Lu-PTHrP cells via subcutaneous injection into the right flank. Starting from the day of tumor cell inoculation animals were administered with different doses of PSP-94 for fifteen consecutive days as described herein. At the end of the study tumors from control (CTL), vehicle treated animals and PSP-94 treated animals were excised and weighed. Control animals receiving vehicle alone exhibited large tumors while treatment with different doses of PSP-94 (0.1-10.0 ug/kg/day) resulted in a significant dose-dependent decrease in tumor weight (FIG. 4).

Inoculation of male Copenhagen rats with Mat Ly Lu-PTHrP cells into the right flank via S.C. injections resulted in the development of primary tumors. Whereas control, vehicle treated animals developed large primary tumors, treatment with different doses of PSP-94 resulted in a dose-dependent decrease in their tumors mass. These anti-tumor effects were not associated with any noticeable side effects or weight loss of experimental animals.

Results presented in Examples 1 to 3 indicate that PSP-94 and PSP-94 fragments (e.g., PCK3145) are not only able to reduce the growth of tumor cells (see U.S. Pat. No. 5,428,011 and Canadian patent application No: 2,359,650) but also reduce the growth of tumor cells having an increased ability to promote tumor progression.

### EXAMPLE 4a

### Effect of PSP-94 on the Development of Skeletal Metastasis.

Since the major cause of prostate cancer related mortality is the development of metastasis, evaluation of the effect of PSP-94 on delaying the development of skeletal metastasis was carried out by inoculating male Copenhagen rats with Mat Ly Lu-PTHrP cells via I.C. route into the left ventricle. Routine injection of Mat Ly Lu cells into the left ventricle results in the development of skeletal metastasis causing compression of the spinal cord leading to hind-limb paralysis.

Thus, male Copenhagen rats were inoculated via I.C. route into the left ventricle with 10×10³ Mat Ly Lu-PTHrP cells. Starting on the time of tumor cell inoculation (day 0) animals were infused with different doses of PSP-94 (0.1-10.0 ug/kg/day) until the day of development of hind-limb paralysis as described herein. Animals receiving vehicle alone as control (CTL) or PSP-94 were monitored daily for the development of hind-limb paralysis and % animals not paralyzed at different time points in each group was calculated.

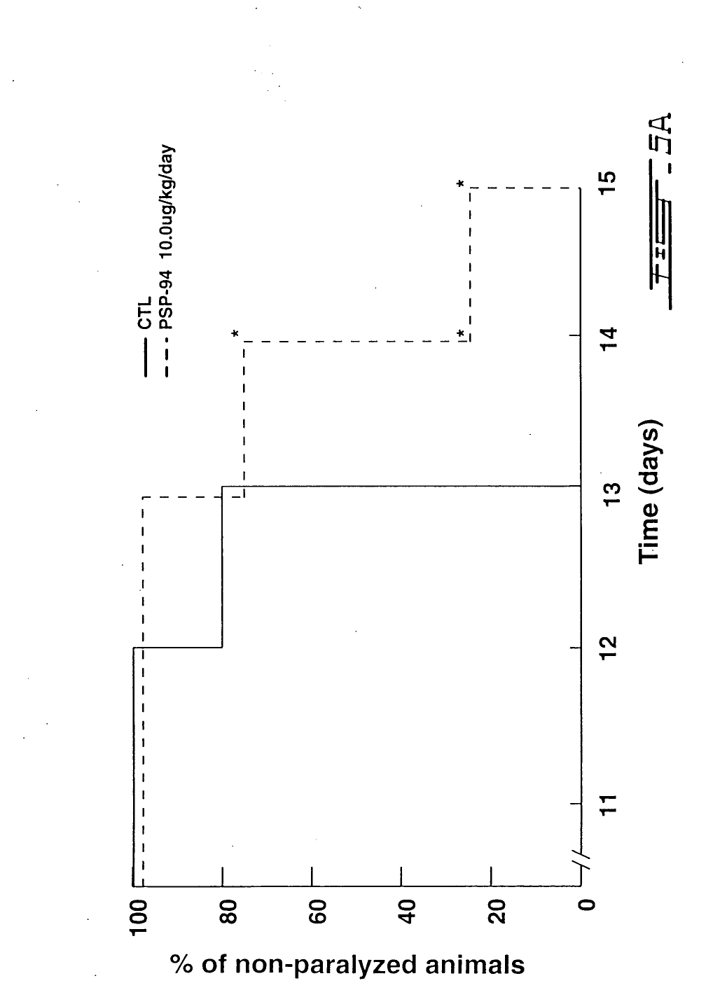

Results presented in FIG. 5A show that every (100%) control animals inoculated with Mat Ly Lu-PTHrP cells and receiving vehicle alone developed hind-limb paralysis by day 13. While 0.1 and 1.0 μg/kg/day PSP-94 had no significant effect on the time of hind limb paralysis (data not shown), treatment with 10.0 μg/kg/day PSP-94 resulted in a statistically significant delay in the number of animals developing hind limb paralysis. Percentage of total number of animals receiving PSP-94 which did not develop hind limb paralysis at different days is shown in FIGS. 5A and 5B.

A second set of experimentation was performed. Results presented in FIG. 5B also show male Copenhagen rats inoculated via the intracardiac (i.c) route but with 5×10⁴ Mat Ly Lu-PTHrP cells. After 3 days of tumor cell inoculation, animals were injected by intraperitoneal route with vehicle alone (Ctl) or different doses of PSP-94 (nPSP). Time to the development of hind limb paralysis in Ctl and animals receiving 10 μg/kg/day of PSP-94 is shown.

While all control, vehicle treated animals developed hind-limb paralysis by day 13 in this experiment, administration of the highest dose of PSP-94 starting from the time of tumor cell inoculation resulted in modest (but measurable) delay in skeletal metastasis. Such results suggest low bioavailability of PSP-94 to the skeleton, a common drawback associated with developing effective therapeutic agents for skeletal metastasis (Rabbani, S. A. et al., Cancer res., 58:3461-3465, 1998).

Therefore, PSP-94 is able to delay the development of metastatic cancer in vivo.

### EXAMPLE4b

### Effect of PCK3145 on the Development of Skeletal Metastasis.

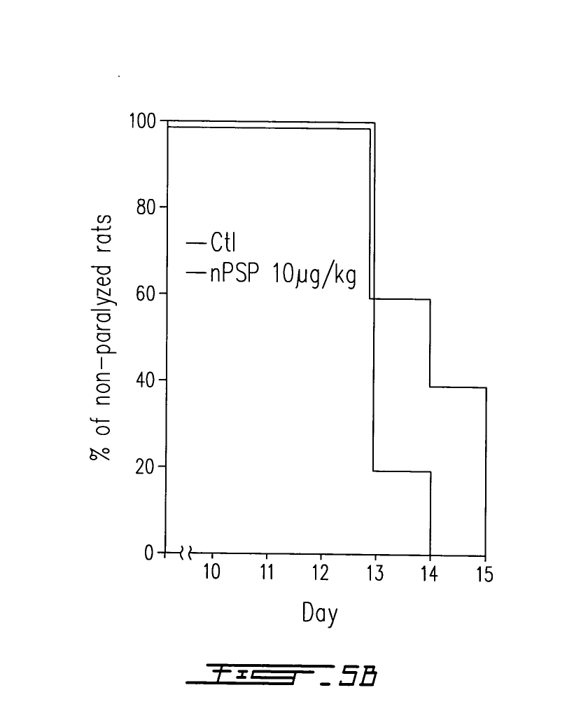

Male Copenhagen rats were inoculated with Mat Ly L u-PTHrP cells (10×10³) into the left ventricle via I.C. injections. Starting on the day of tumor cell inoculation (day 0), experimental animals received different doses of PCK3145 (1.0-100.0 ug/kg/day) via I.P. route. To evaluate the effect of PCK3145 on delaying the development of experimental skeletal metastasis, animals were evaluated daily by monitoring of the animals for the development of hind-limb paralysis. All (100%) control animals inoculated with Mat Ly Lu-PTHrP cells and receiving vehicle alone developed hind-limb paralysis by day 13. Treatment with 1.0 and 10.0 μg/kg/day PCK3145 had no significant effect on delaying the time of developing hind limb paralysis (data not shown). In contrast, treatment with 100.0 μg/kg/day of PCK3145 resulted in a statistically significant delay in the number of animals developing hind limb paralysis. Percentage of total number of animals not developing hind limb paralysis at different days is shown in FIG. 6. Therefore, PCK3145 is able to delay the development of metastatic cancer in vivo.

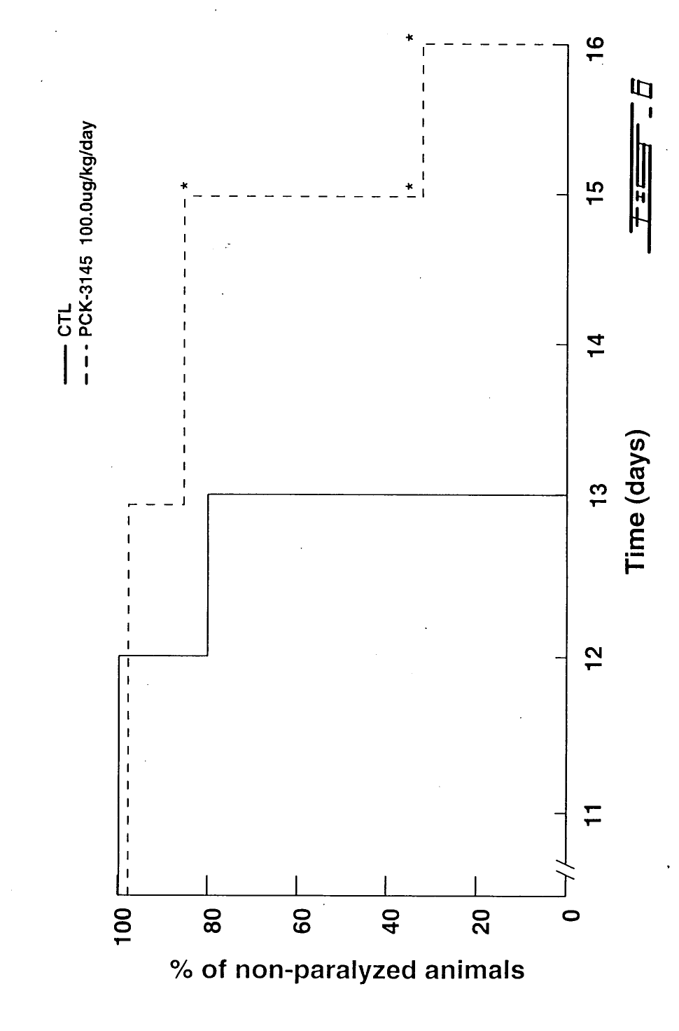

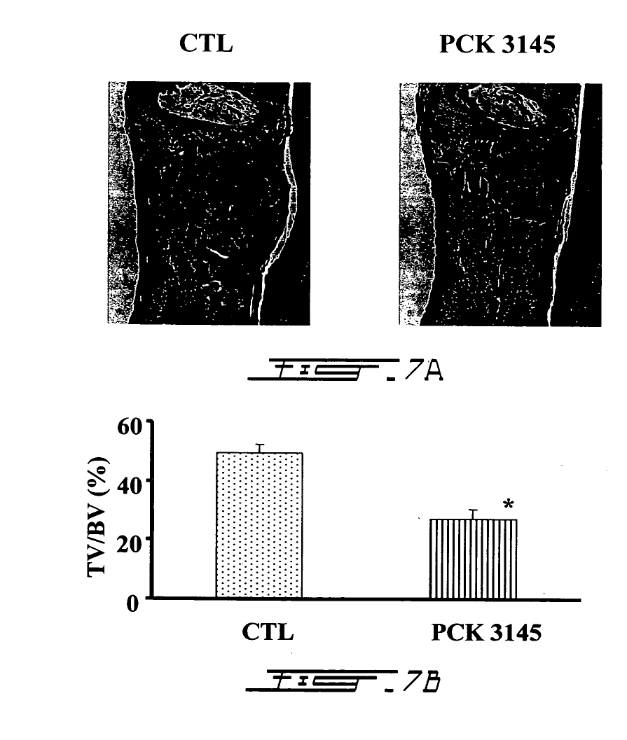

In order to determine the effect of PCK3145 on skeletal tumor burden, similar experiments were performed but analyses were made before the apparition of hind limb paralysis. Briefly, control and experimental animals (Male Copenhagen rats) were infused with vehicle alone or the highest dose of PCK3145 (100.0 ug/kg/day) starting from the day of tumor cell (1×10³ Mat Ly lu-PTHrP cells) inoculation for up to 10 days post tumor cell inoculation upon which they were sacrificed (i.e., when these animals do not exhibit any sign of hind limb paralysis). Vertebra from control (CTL) and experimental animals were removed, decalcified and subjected to bone histomorphometric analysis. Histologic sections of vertebrae from animals receiving vehicle alone or PCK3145 were also stained with H & E as described herein. A significant decrease in the ratio of tumor volume to bone volume was observed in experimental animals receiving 100.0 μg/kg of PCK3145 (FIG. 7A and 7B). In the vertebrae of control animals, clear evidence of both trabecuar and cortical bone destruction was seen which resulted in the compression of the spinal cord (SC) by the tumor (T) causing hind limb paralysis at later time points. In contrast these effects were significantly less in experimental animals receiving PCK3145. FIG. 7B illustrates the same results expressed as the ratio of tumor volume to bone volume (TV/BV) which was determined as described herein. These results clearly show the full effectiveness of PCK3145 on reducing tumor burden in the skeleton before the apparition of hind limb paralysis is noticeable.

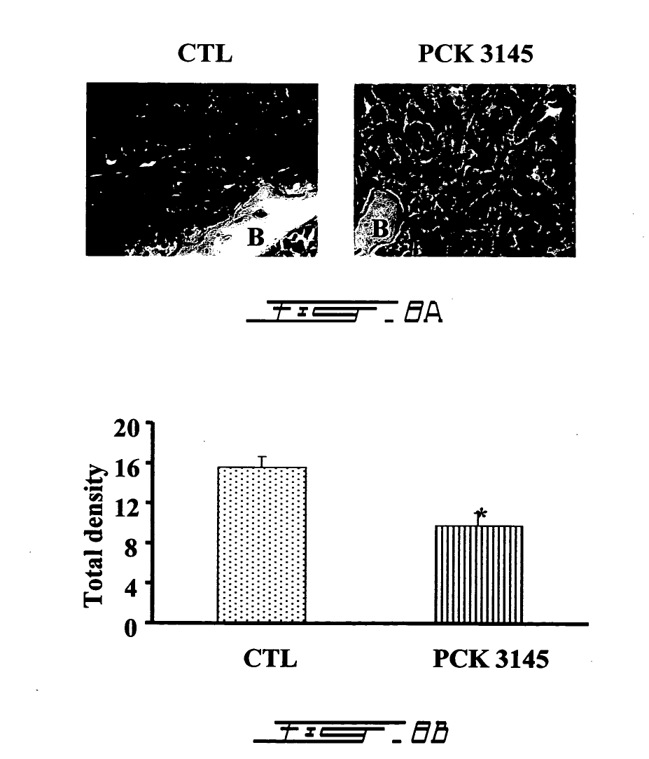

Histologic sections of vertebrae from animals receiving vehicle alone or the highest dose of PCK3145 were also subjected to immunohistochemical analyses using an antibody directed against PTHrP (1-34) as described herein. Representative photomicrograph of three such experiments is shown in FIG. 8A. Levels of PTHrP production in control and experimental metastasis were quantified and expressed as total density (FIG. 8B). Control vertebrae from animals receiving vehicle alone showed strong staining for PTHrP which was significantly lower in Mat Ly Lu-PTHrP cells present in the vertebral column of animals treated with 100.0 μg/kg of PCK3145 (FIG. 8A, 8B). Significant differences in the levels of PTHrP production from control tumor-bearing animals receiving vehicle alone are represented by asterisks (p<0.05). These results indicate that PCK3145 (a PSP-94 fragment) is able to reduce the level of PTHrP expression and/or production in tumor cells before the apparition of hind limb paralysis is noticeable.

### EXAMPLE5a

### Effect of PSP-94 on Plasma PTHrP and Calcium Levels and Tumoral PTHrP Production.

In order to determine the effect of PSP-94 and PCK3145 on plasma PTHrP and calcium levels animals inoculated with Mat Ly Lu-PTHrP cells (1×10⁶ cells) via S.C. route were sacrificed at the end of the study (day 16) (see Example 4a), plasma was collected from control (CTL) vehicle treated animals and PSP-94 treated animals and analyzed for PTHrP levels using a radioimmunoassay. Comparison was made between plasma collected from normal, non-tumor bearing animals (N), control tumor bearing animals receiving vehicle alone (CTL) and plasma collected from experimental animals receiving different doses of PSP-94 (0.1-10.0 ug/kg/day). Plasma calcium levels were determined by atomic absorption spectrophotometry (model 703, Perkin-Elmer, Norwalk, Conn.). For plasma PTHrP, all samples were tested in two dilutions in PTHrP R.I.A. kit (Nichols Institute Diagnostics, San Juan Capistrano, Calif.) according to manufacturers instructions.

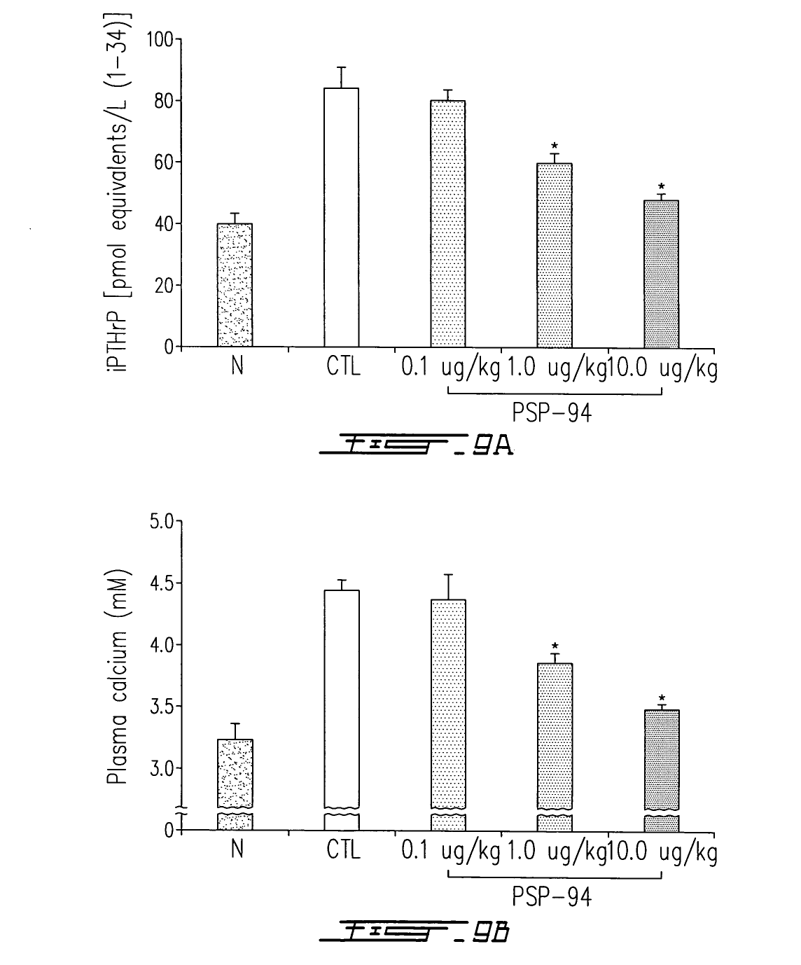

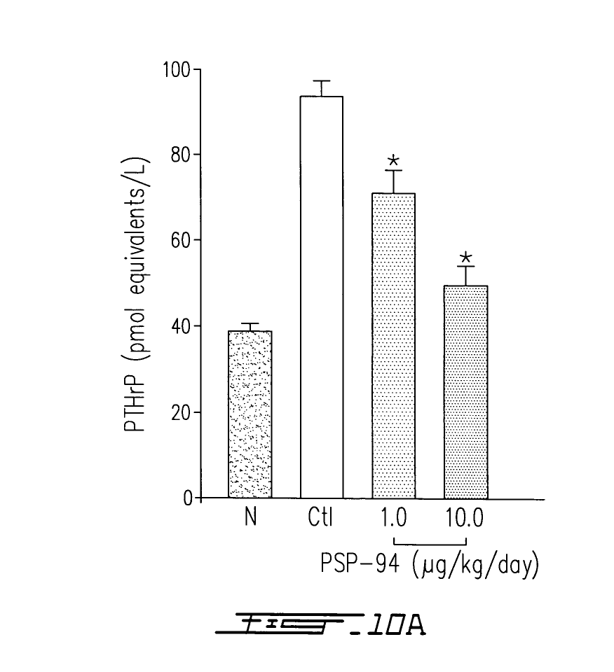

Results presented in FIG. 9A and 9B indicate that normal non-tumor bearing animals exhibited basal levels of plasma PTHrP whereas animals inoculated with Mat Ly Lu-PTHrP cells and receiving vehicle alone showed marked elevated levels of immunoreactive plasma PTHrP levels. Treatment of tumor bearing animals with PSP-94 resulted in a dose-dependent decrease in plasma PTHrP levels (FIG. 9A). Analysis of plasma collected from normal non-tumor bearing animals and tumor bearing animals receiving vehicle alone revealed a marked increase in plasma calcium of control tumor bearing animals at the time of sacrifice on day 16 post-tumor cell inoculation. In contrast, experimental groups of animals receiving different doses of PSP-94 resulted in significant reduction in their plasma calcium levels. The highest dose of PSP-94 (10.0 ug/kg/day) resulted in near normalization of plasma calcium of these experimental group of animals (FIG. 9B).

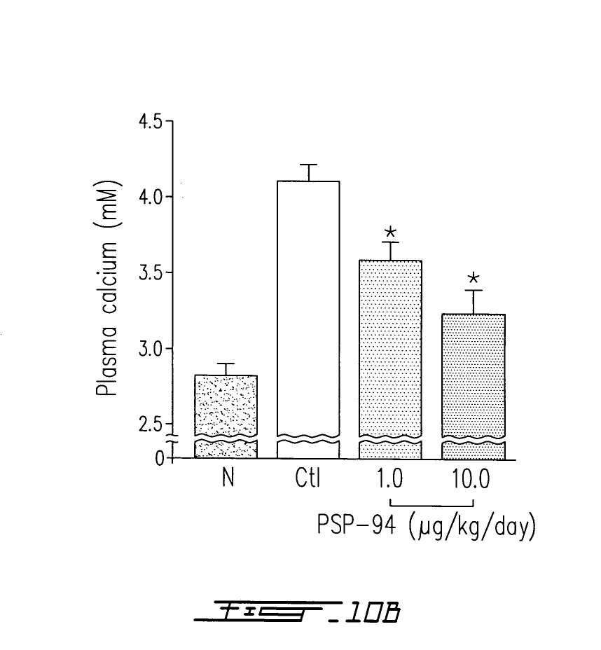

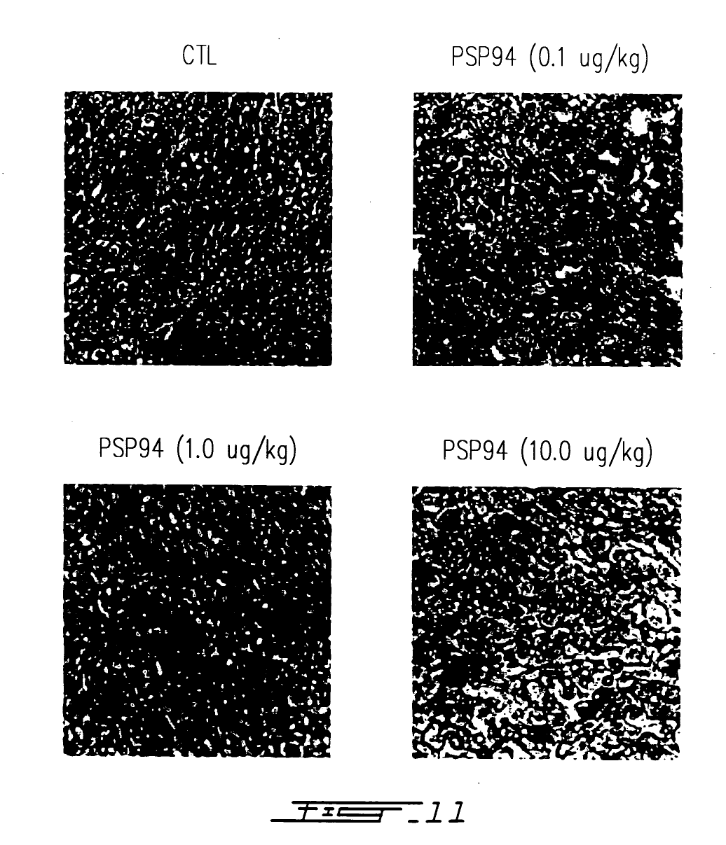

A second set of experiment was performed and similar results were obtained. FIGS. 10A and 10B show male Copenhagen rats inoculated s.c. with 10⁶ Mat Ly Lu-PTHrP cells. Following 3 days of tumor cell inoculation, animals were treated with vehicle alone (Ctl) or different doses (1.0, or 10.0 ug/kg/day) of PSP-94 for 18 days. Animals were sacrificed on day 21 and plasma PTHrP (FIG. 10A; expressed in picomole equivalents/liter) or plasma calcium (FIG. 10B; expressed in millimolar (mM)) levels were determined. Results for non-tumor bearing animals are also shown (N).

These results indicate that normal, non-tumor bearing animals have undetectable levels of plasma PTHrP whereas inoculation of animals with Mat Ly Lu-PTHrP cells resulted in marked increase in their plasma PTHrP levels. In contrast to this, treatment with the different doses of PSP-94 resulted in a dose-dependent decrease in plasma PTHrP levels. In addition, the same dose-dependent decrease was observed in tumoral PTHrP production when tumor samples from control, vehicle treated and PSP-94 treated animals were subjected to immunohistochemical analysis.

Inoculation of Mat Ly Lu-PTHrP cells into the animals resulted in a marked increase in their plasma calcium levels as compared to serum from normal, non-tumor bearing animals. Administration of different doses of PSP-94 resulted in a dose-dependent decrease in plasma calcium levels with the highest dose of PSP-94 leading to a near normalization of plasma calcium levels. Being the major pathogenetic factor of hypercalcemia of malignancy, plasma calcium levels correlate with that of plasma PTHrP levels (Iwamura, M., et al., Urology 43:675-679, 1994; Iwamura, M., et al., Hum. Pathol. 26:797-801, 1995; Suva, L. J., et al., Science, 237:893-896,1987).

Tumors from control group treated with vehicle alone and experimental groups treated with different doses of PSP-94 (0.1-10.0 μg/kg/day) were excised, paraffin embedded, and sectioned and analyzed for tumoral PTHrP production by immunohistochemical reaction specific for PTHrP using method described herein. Results of FIG. 11 show histological sections of tumors from animals receiving vehicle alone (CTL) or different doses of PSP-94 and stained with an antibody specific for PTHrP as described herein. Three animals were present in each group and three tumor sections were analyzed for each animal by evaluating at least ten random fields of observation.

Intense color staining of tumors from control groups of animals receiving vehicle alone was observed. In contrast a dose dependent decrease in PTHrP immunostaining was observed in experimental tumors from animals receiving different doses of PSP-94 (FIG. 11).

### EXAMPLE 5b

### Effect of PCK3145 on Plasma PTHrP and Calcium Levels and Tumoral PTHrP Production.

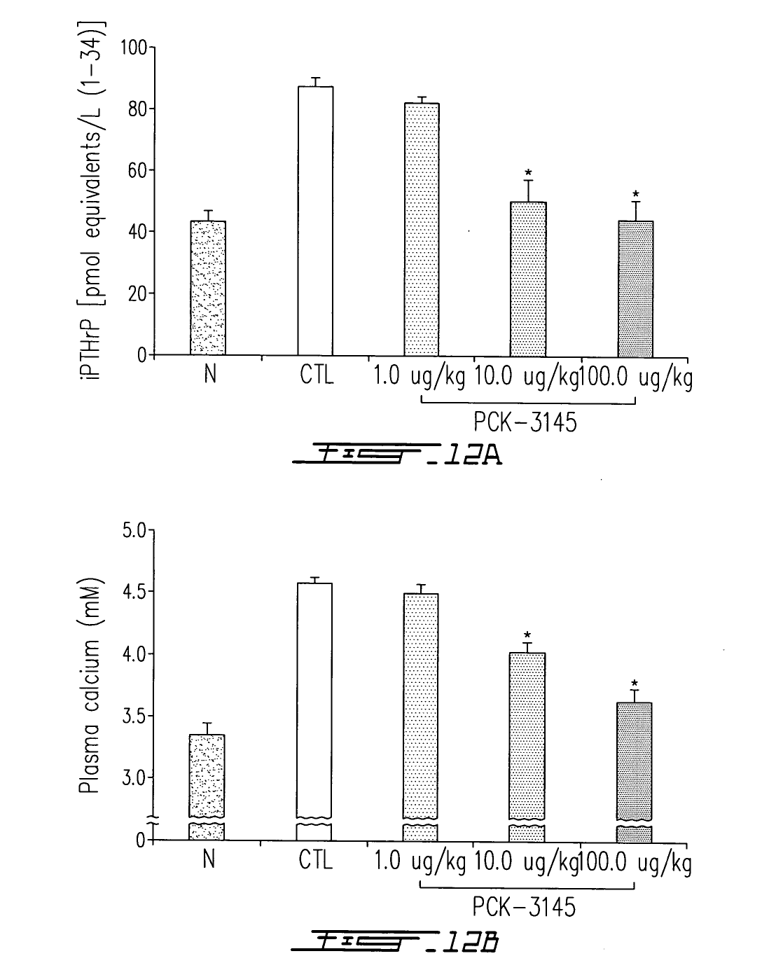

In order to determine the effect of PCK3145 on plasma PTHrP and calcium levels, animals inoculated with 1×10⁶ Mat Ly Lu-PTHrP cells via S.C. route were sacrificed at the end of the study (day 16) (see Example 4b), plasma was collected and PTHrP levels were analyzed using a radioimmunoassay. Comparison was made between plasma collected from normal, non-tumor bearing animals, control tumor bearing animals receiving vehicle alone and experimental animals receiving different doses of PCK3145 (1.0-100.0 ug/kg/day). Normal non-tumor bearing animals showed basal levels of plasma PTHrP whereas animals inoculated with Mat Ly Lu-PTHrP cells and receiving vehicle alone showed marked elevated levels of immunoreactive plasma PTHrP levels. Treatment of tumor bearing animals with PCK3145 resulted in a dose-dependent decrease in plasma PTHrP levels (FIG. 12A). Plasma PTHrP levels in normal non-tumor bearing animals are also shown (N).

Analysis of plasma collected from normal non-tumor bearing animals and tumor bearing animals receiving vehicle alone revealed a marked increase in plasma calcium levels of control tumor bearing animals at the time of sacrifice on day 16 post tumor cell inoculation. In contrast, experimental groups of animals receiving different doses of PCK3145 exhibited a significant reduction in their plasma calcium levels; effects which were more pronounced in experimental animals receiving 100.0 μg/kg of PCK3145 (FIG. 12B). Plasma calcium levels from non-tumor bearing animals are also shown (N).

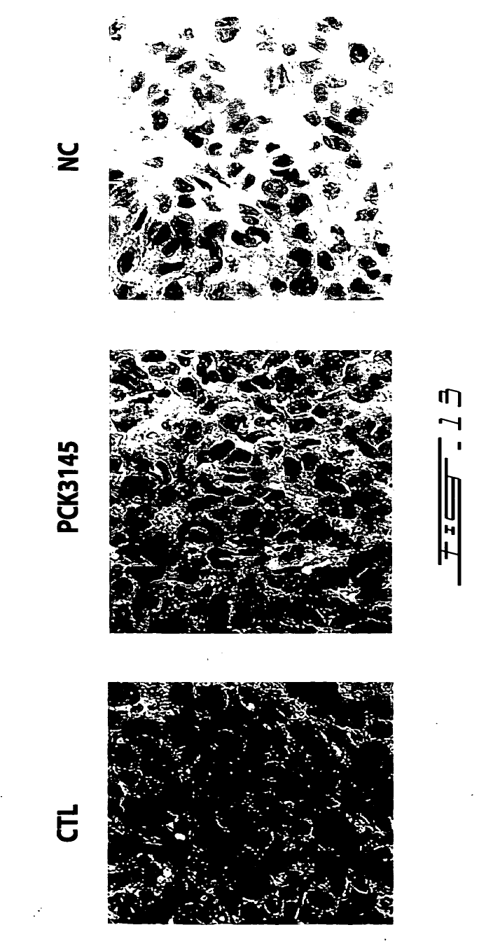

Primary tumors from control vehicle treated animals and animals treated with the highest dose of PCK3145 (100.0 ug/kg/day) were removed, paraffin embedded, and sectioned. Histologic sections of tumors from animals receiving vehicle alone (CTL) or the highest dose of PCK3145 were stained with an antibody directed against PTHrP (1-34) as described herein. These results are presented in FIG. 13. Also shown is a negative control (NC) where primary antibody was not used to account for background staining. Therefore, tumors from control group treated with vehicle alone and experimental group treated with the highest dose of PCK3145 (100.0 ug/kg/day) were excised and analyzed for tumoral PTHrP production by immunohistochemical analysis. Intense PTHrP staining of tumor cells from control groups of animals receiving vehicle alone was observed (CTL). In contrast a dose dependent decrease in PTHrP immuno-staining was observed in experimental tumors from animals receiving different doses of PCK3145 (data not shown). These effects were most pronounced in tumors from animals receiving 100.0 μg/kg of PCK3145. No reaction was seen when these tumors were evaluated for background reaction in the absence of PTHrP antibody (NC). These results confirm that PCK3145 is able to reduce tumoral PTHrP production.

### EXAMPLE 6

### Effect of PSP-94 Mat Ly Lu-PTHrP Tumor Cell Apoptosis in vitro and in vivo.

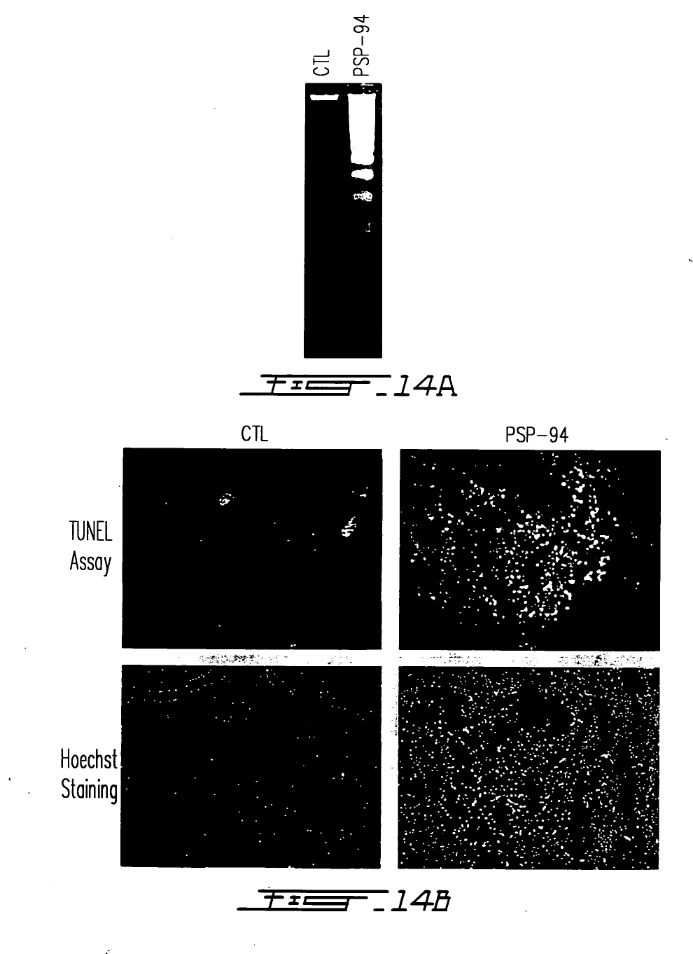

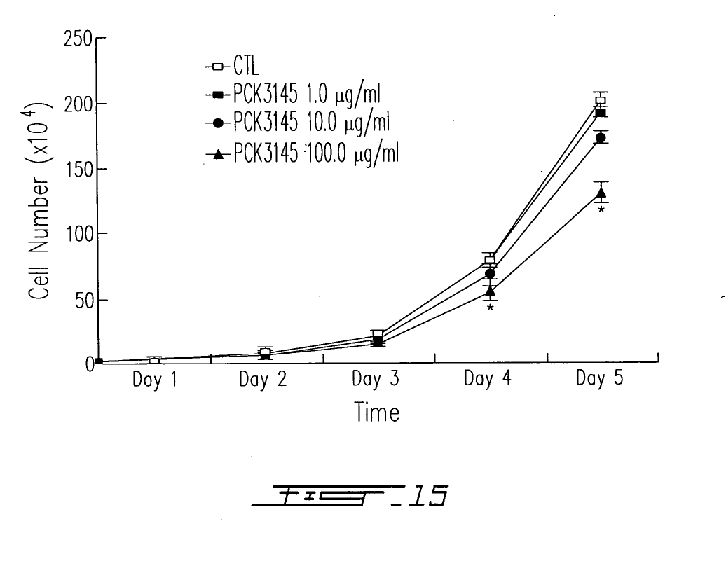

In order to investigate the underlying molecular mechanism of action of PSP-94 in reducing tumor growth Mat Ly Lu-PTHrP cells were cultured in the presence of PSP-94 (10.0 ug/ml) or vehicle alone for different time intervals. Genomic DNA was collected from cells cultured in the presence of vehicle alone or PSP-94. Briefly, for DNA fragmentation, Mat Ly Lu-PTHRP cells were plated in 6 well plates (Falcon Plastics, Oxnard, Calif.). Cells were treated with PSP-94 (10.0 ug/ml) for up to 72 hours. DNA from treated cells incubated with PSP-94 and cells treated with vehicle alone was collected using a Phenol:Choloroform:lsoamyl alcohol solution (50:48:2). Equal amounts of DNA were subjected to gel electrophoresis on a 2% agarose gel. DNA fragmentation was visualised by UV light using a transilluminator. More particularly, results presented in FIG. 14A show Mat Ly Lu-PTHrP cells cultured in the presence of vehicle alone or PSP-94 (10.0 ug/ml) for up to 96 hours. DNA was isolated and run in an agarose gel, as described above. These results show that control Mat Ly Lu-PTHrP cells cultured with vehicle alone exhibited no signs of DNA fragmentation. However, experimental Mat Ly Lu-PTHrP cells cultured in the presence of PSP-94 (10.0 ug/ml) exhibited marked DNA fragmentation after 72 hours of treatment (FIG. 14A).

The degree of DNA fragmentation was also analyzed in vivo using TUNEL assay which can serve as a marker for apoptosis. More particularly, results presented in FIG. 14B are derived from tissue collected from Male Copenhagen rats inoculated with 1×10⁶ Mat Ly Lu-PTHrP cells and infused with different doses of PSP-94 for fifteen consecutive days as described herein. All animals were sacrificed at the end of the study and their primary tumors removed, paraffin embedded, sectioned and processed by TUNEL assay (upper panel of FIG. 14B) as described herein. Following TUNEL assay, they were counterstained with Hoescht reagent (lower panel of FIG. 14B). Tumor sections treated with PSP-94 (10.0 ug/kg/day) were significantly more TUNEL positive as compared to vehicle treated control tumors (FIG. 14B). Counterstaining with Hoechst reagent revealed the presence of apoptotic bodies in tissue sections from animals treated with PSP-94. Furthermore, condensed chromatin, which is characteristic of apoptotic cells, was observed in PSP-94 treated tumors. Control, vehicle treated tumors exhibited normal DNA staining patterns (FIG. 14B). These in vitro and in vivo findings demonstrate that indeed reduction in tumor volume following treatment with PSP-94 which may be due to its ability to promote tumor cell apoptosis.

### EXAMPLE 7

### Effect of PCK3145 on MatLyLu-PTHrP Cell Growth and Morphology.

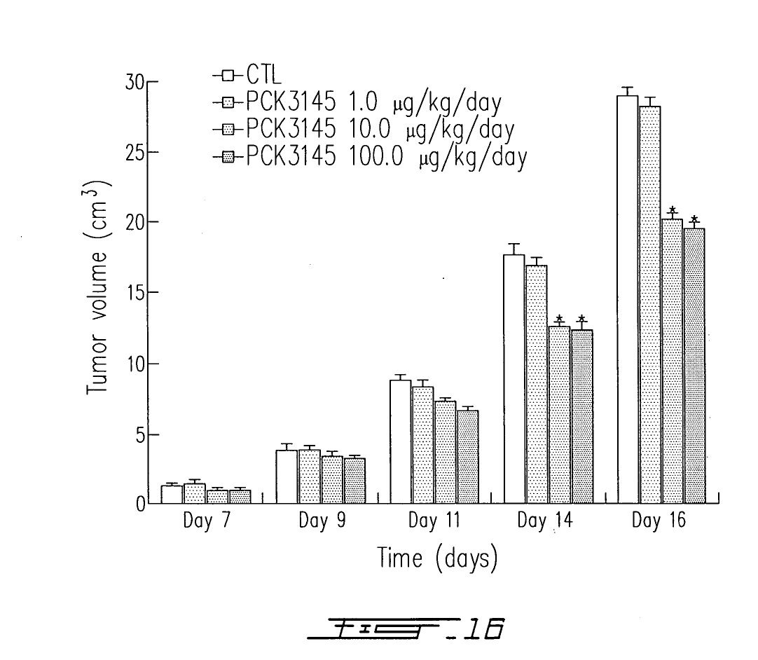

Mat Ly Lu-PTHrP cells were seeded at a density of 5×10³ cells/well in 6-well plates and treated with different doses (1.0-100.0 ug/ml) of PCK3145 for up to 5 days and were trypsinized and counted using a coulter counter as described herein. Treatment with PCK3145 resulted in a dose dependent decrease in cell growth with the highest dose (100.0 ug/ml) exhibiting a significant reduction in cell number compared to control Mat Ly Ly-PTHrP cells treated with vehicle alone (FIG. 15). In addition, Mat Ly Lu-PTHrP cells treated with 100.0 ug/ml of PCK3145 changed their morphology from a more spindle-like appearance to a more circular and condensed shape (data not shown).

### EXAMPLE 8

### Effect of PCK3145 on Mat Ly Lu-PTHrP Tumor Growth and Weight.

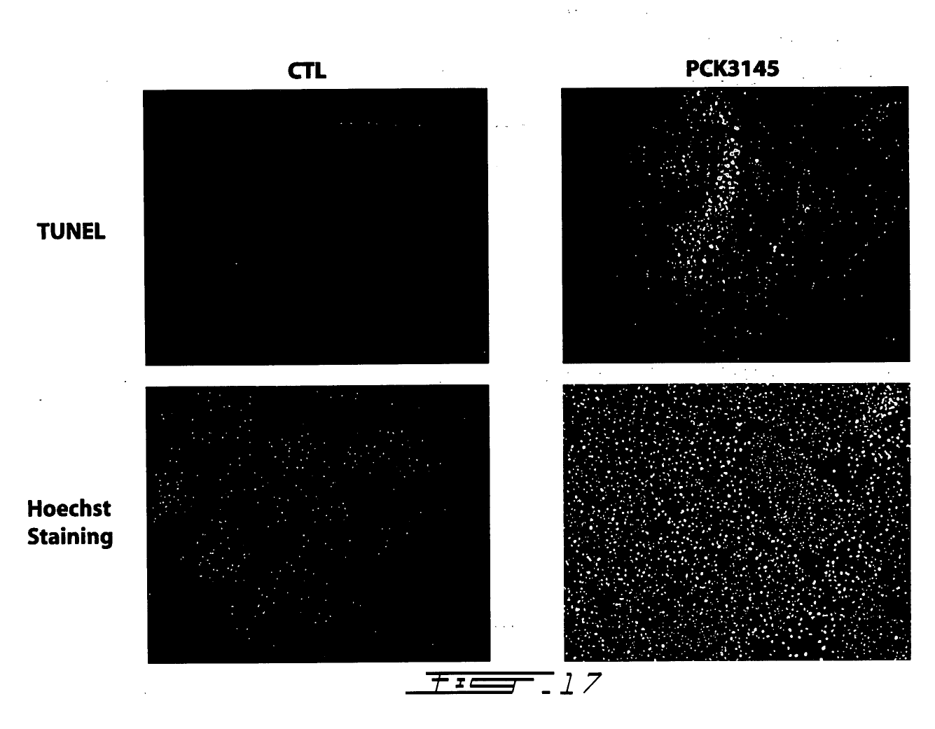

Male Copenhagen rats were inoculated with 1×10⁶ Mat Ly Lu-PTHrP cells via S.C. route of injection into the right flank. Animals were infused daily via S.C. route, below the site of tumor cell inoculation, with different doses (1.0-100.0 ug/kg/day) of PCK3145 starting from the day of tumor cell inoculation and lasting for 15 days. The effect of PCK3145 on reducing tumor growth was evaluated by daily determination of tumor volume using and comparison was made with control tumor-bearing animals receiving vehicle alone. A progressive increase in tumor size was seen in control animals throughout the duration of the study. Experimental animals receiving PCK3145 showed a significant dose-dependent reduction in tumor volume throughout the course of the study (FIG. 16) compared to control animals (CTL). Animals were also weighed to determine any side effects of administering PCK3145. No significant difference in animal weight was observed that can be attributed to any side effects of receiving PCK3145. In order to determine the effect of PCK3145 on tumor weight, control animals and experimental animals receiving PCK3145 were sacrificed at the end of the study (day 16) and their tumors excised and weighed. Treatment with the different doses of PCK3145 resulted in a dose-dependent decrease in tumor weight as compared to control tumors excised from animals receiving vehicle alone (data not shown).

### EXAMPLE 9

### Effect of PCK3145 on Mat Ly Lu-PTHrP Tumor Cell Apoptosis in vivo.

In order to investigate an underlying molecular mechanism of action of PCK3145 in reducing tumor growth, Mat Ly Lu-PTHrP tumors were analyzed using TUNEL assay which can serve as a marker for apoptosis. Briefly, male Copenhagen rats were inoculated with 1×10⁶ Mat Ly Lu-PTHrP cells. Starting on the time of tumor cell inoculation animals were infused with different doses of PCK3145 for fifteen consecutive days as described herein. All animals were sacrificed at the end of the study and their primary tumors removed, paraffin embedded, sectioned and processed by TUNEL assay (upper panel) and counterstained with Hoescht reagent (lower panel). Five animals were present in each group and three sections were analyzed for each animal. Tumor sections treated with PCK3145 (100.0 ug/kg/day) were significantly more TUNEL positive as compared to vehicle treated control tumors (FIG. 17). Counterstaining with Hoechst reagent revealed the presence of apoptotic bodies in tissue sections from experimental animals treated with PCK3145. Furthermore, condensed chromatin, a feature characteristic of apoptotic cells, was observed in PSP-94 treated tumors. Control, vehicle treated tumors exhibited normal DNA staining patterns (FIG. 17). These in vivo findings demonstrate that indeed reduction in tumor volume following treatment with PCK3145 may be due to its ability to promote tumor cell apoptosis.

Using the model presented herein, the anti-tumor effects of PSP-94 and PCK3145 in the reduction in tumor volume of skeletal metastasis was demonstrated as well as biochemical parameters like plasma calcium and PTHrP levels showed a marked decrease following therapy. A significant finding in the present studies was that while decrease in tumor volume was dose-dependent, 10.0 ug/kg/day PSP-94 did not show a marked decrease in tumor volume as compared to 1.0 ug/kg/day PSP-94. In contrast, the ability of PSP-94 to reduce plasma calcium, plasma PTHrP and tumoral PTHrP continued to show a dose-dependent effect with 10.0 ug/kg/day PSP-94 causing near normalization of plasma calcium and PTHrP levels. These findings indicate that PSP-94 may also have additional effects including its ability to regulate PTHrP production by tumor cells or alter calcium homeostasis. Indeed PSP-94 has been shown to suppress follicle stimulating hormone (FSH) which is known to regulate intracellular calcium (Touyz, R. M. et al., Biol. Reprod. 62:1067-1074, 2000). Suppression of FSH by PSP-94 may serve as an additional mechanism to cause anti-tumor effects due to the growth-promoting effects of FSH in prostate cancer (Porter, A. T. et al., Urol. Oncol., 6:131-138,2001). Furthermore, although cloning and characterization of a putative PSP-94 receptor has not been performed several studies have provided evidence for the existence of PSP-94 binding proteins on prostate cancer cells which could allow PSP-94 binding to initiate a signaling cascade that results in the observed anti-tumor effects observed (Yang, J. P. et al., J.Urol. 160:2240-2244, 1998; Yang, J. P. et al., Prostate, 35:11-17, 1998).

Collectively, the results of this study demonstrate PSP-94 and PCK3145 to be effective inhibitors of hormone-independent, late stage prostate cancer growth and its associated hypercalcemia of malignancy without manifesting any noticeable cytotoxic effects. The use of polypeptides, their peptidomimetic analogues alone or in combination with currently available chemotherapeutic agents will provide unique opportunities to block prostate cancer progression with highly effective non-toxic biotherapeutic agents which can be delivered over a long period of time without any drug associated side effects. These approaches will go a long way in reducing prostate cancer associated morbidity and mortality.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for treating a patient having a condition selected from the group consisting of a metastasis, a metastatic cancer, a condition associated with elevated levels of parathyroid hormone-related protein, PTHrP-induced osteolysis and hypercalcemia of malignancy, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof:

2. The method as defined in claim 1, wherein said condition is selected from the group consisting of a metastatic prostate cancer, a metastatic breast cancer, a metastatic lung cancer, a metastatic hepatocellular cancer and a metastatic myeloma.

3. The method as defined in claim 2, wherein said metastatic prostate cancer is a late stage hormone refractory metastatic prostate cancer.

4. The method as defined in claim 1, wherein said metastasis is a skeletal metastasis.

5. The method as defined in claim 1, wherein said compound is SEQ ID NO.:5.

6. The method as defined in claim 4, wherein said compound is a SEQ ID NO.:5 biologically active analogue having an anti-metastatic effect.

7. A compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof for use in the treatment of a patient having a condition selected from the group consisting of metastasis, metastatic cancer, a condition associated with elevated levels of parathyroid hormone-related protein, PTHrP-induced osteolysis and hypercalcemia of malignancy.

8. The compound of claim 7, wherein said condition is selected from the group consisting of a metastatic prostate cancer, a metastatic breast cancer, a metastatic lung cancer, a metastatic hepatocellular cancer and a metastatic myeloma.

9. The compound as defined in claim 8, wherein said metastatic prostate cancer is a late stage hormone refractory metastatic prostate cancer.

10. The compound as defined in claim 7, wherein said metastasis is a skeletal metastasis.

11. The compound as defined in claim 7, wherein said compound is SEQ ID NO.:5.

12. The compound as defined in claim 10, wherein said compound is a SEQ ID NO.:5 biologically active analogue having an anti-metastatic effect.

13. A pharmaceutical composition for use in the treatment of a condition selected from the group consisting of metastasis, metastatic cancer, a condition associated with elevated levels of parathyroid hormone-related protein, PTHrP-induced osteolysis and hypercalcemia of malignancy, the pharmaceutical composition comprising a compound as defined in claim 7 and a pharmaceutically acceptable carrier.

14. A method for reducing the levels of PTHrP in a mammalian cell, the method comprising contacting the mammalian cell with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

15. The method as defined in claim 14, wherein said compound is SEQ ID NO.:5.

16. The method as defined in claim 14, wherein said compound is a SEQ ID NO.:5 biologically active analogue having an anti-metastatic effect.

17. A method of manufacturing a medicament for the treatment of a patient having a condition selected from the group consisting of metastasis, metastatic cancer, a condition associated with elevated levels of parathyroid hormone-related protein, PTHrP-induced osteolysis and hypercalcemia of malignancy, the method comprising the steps of;
a) obtaining a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof, and;
b) combining said compound with a pharmaceutically acceptable carrier or excipient.

18. The method of claim 17, wherein said condition is selected from the group consisting of a metastatic prostate cancer, a metastatic breast cancer, a metastatic lung cancer, a metastatic hepatocellular cancer and a metastatic myeloma.

19. The method as defined in claim 18, wherein said metastatic prostate cancer is a late stage hormone refractory metastatic prostate cancer.

20. The method as defined in claim 17, wherein said metastasis is skeletal metastasis.

21. The method as defined in claim 17, wherein said compound is SEQ ID NO.:5.

22. The method as defined in claim 20, wherein said compound is a SEQ ID NO.:5 biologically active analogue having an anti-metastatic effect.

23. The use of a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof in the manufacture of a medicament for the treatment of a patient having a condition selected from the group consisting of metastasis, metastatic cancer, a condition associated with elevated levels of parathyroid hormone-related protein, PTHrP-induced osteolysis and hypercalcemia of malignancy.

24. The use as defined in claim 23, wherein said condition is selected from the group consisting of a metastatic prostate cancer, a metastatic breast cancer, a metastatic lung cancer, a metastatic hepatocellular cancer and a metastatic myeloma.

25. The use as defined in claim 24, wherein said metastatic prostate cancer is a late stage hormone refractory metastatic prostate cancer.

26. The use as defined in claim 19, wherein said metastasis is a bone metastasis.

27. The use as defined in claim 23, wherein said compound is SEQ ID NO.:5.

28. The use as defined in claim 26, wherein said compound is a SEQ ID NO.:5 biologically active analogue having an anti-metastatic effect.

29. A method for evaluating, the efficacy of a treatment of a patient having a condition selected from the group consisting of bone metastasis, metastatic cancer, elevated levels of parathyroid hormone-related protein, PTHrP-induced osteolysis and hypercalcemia of malignancy, the treatment of a patient being a treatment with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof, the method comprising measuring a plasma calcium level of the patient after the treatment.

30. The method as defined in claim 29, wherein the plasma calcium level of the patient after the treatment is compared with a plasma calcium level of the patient before the treatment.

31. The method of claim 30, wherein said plasma calcium levels are measured from a plasma sample of a patient.

32. The method of claim 29, wherein said condition is selected from the group consisting of a metastatic prostate cancer, a metastatic breast cancer, a metastatic lung cancer, a metastatic hepatocellular cancer and a metastatic myeloma.

33. A method of manufacturing a pharmaceutical composition for the treatment of a patient having a condition selected from the group consisting of metastasis, metastatic cancer, elevated levels of parathyroid hormone-related protein, PTHrP-induced osteolysis and hypercalcemia of malignancy, the method comprising the steps of;
a) obtaining a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof, and;
b) combining said compound with a pharmaceutically acceptable carrier or excipient.

34. The method as defined in claim 33, wherein said condition is selected from the group consisting of a metastatic prostate cancer, a metastatic breast cancer, a metastatic lung cancer, a metastatic hepatocellular cancer and a metastatic myeloma.

35. The method as defined in claim 34, wherein said metastatic prostate cancer is a late stage hormone refractory metastatic prostate cancer.

36. The method as defined in claim 33, wherein said metastasis is skeletal metastasis.

37. The method as defined in claim 33, wherein said compound is SEQ ID NO.:5.

38. The method as defined in claim 36, wherein said compound is a SEQ ID NO.:5 biologically active analogue having an anti-metastatic effect.

39. A method for reducing tumor cell invasion or spreading of tumor cells to distant sites, the method comprising administering to a patient in need thereof, a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

40. A method for reducing the growth of a metastatic cancer cell, the method comprising contacting the metastatic cancer cell with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

41. The method of claim 40, wherein said metastatic cancer cell is a metastatic bone cancer cell.

42. The method of claim 41, wherein said metastatic bone cancer cell is from a cancer selected from the group consisting of a metastatic prostate cancer, a metastatic breast cancer, a metastatic lung cancer, a metastatic hepatocellular cancer and a metastatic myeloma.

43. The method as defined in claim 40, wherein said compound is SEQ ID NO.:5.

44. The method as defined in claim 40, wherein said compound is a SEQ ID NO.:5 biologically active analogue having an anti-metastatic effect.

45. The method of claim 40, wherein said metastatic cancer cell is expressing PTHrP.

46. The use of a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof for reducing the growth of a metastatic cancer cell.

47. The use as defined in claim 46, wherein said metastatic cancer cell is a metastatic bone cancer cell.

48. The use as defined in claim 47, wherein said metastatic bone cancer cell is from a cancer selected from the group consisting of a metastatic prostate cancer, a metastatic breast cancer, a metastatic lung cancer, a metastatic hepatocellular cancer and a metastatic myeloma.

49. The use as defined in claim 46, wherein said compound is SEQ ID NO.:5.

50. The use as defined in claim 46, wherein said compound is a SEQ ID NO.:5 biologically active analogue having an anti-metastatic effect.

51. The use as defined in claim 46, wherein said metastatic cancer cell is expressing PTHrP.

52. A pharmaceutical composition for inhibiting the growth of a metastatic cancer cell, the pharmaceutical composition comprising a compound as defined in claim 7 and a pharmaceutically acceptable carrier.

53. The composition as defined in claim 52, wherein said metastatic cancer cell is a metastatic bone cancer cell.

54. The composition as defined in claim 53, wherein said metastatic bone cancer cell is from a cancer selected from the group consisting of a metastatic prostate cancer, a metastatic breast cancer, a metastatic lung cancer, a metastatic hepatocellular cancer and a metastatic myeloma.

55. The composition as defined in claim 52, wherein said compound is as defined in SEQ ID NO:5.

56. The composition as defined in claim 52, wherein said compound is a SEQ ID NO.:5 biologically active analogue having an anti-metastatic effect.

57. The composition as defined in claim 52, wherein said metastatic cancer cell is expressing PTHrP.

58. A method for reducing the expression of PTHrP in a cancer cell, the method comprising contacting the cancer cell with a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

59. The method of claim 58, wherein said cancer cell is selected from the group consisting of a prostate cancer cell, a breast cancer cell, a lung cancer cell, a liver cell and a bone marrow cell.

60. The method of claim 59, wherein said prostate cancer cell is a metastatic prostate cancer cell.

61. A pharmaceutical composition for use in the reduction of the development of metastasis, the pharmaceutical composition comprising a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof, and a pharmaceutically acceptable carrier.

62. The pharmaceutical composition of claim 61, wherein said metastasis is a skeletal metastasis.

63. The pharmaceutical composition of claim 61, wherein said compound is SEQ ID NO.:5.

64. The pharmaceutical composition of claim 61, wherein said compound is a SEQ ID NO.:5 biologically active analogue having an anti-metastatic effect.

65. A method for reducing the development of metastasis, the method comprising providing to a metastatic cancer cell, a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

66. The method of claim 65, wherein said compound is SEQ ID NO.:5.

67. The method of claim 65, wherein said compound is a SEQ ID NO.:5 biologically active analogue having an anti-metastatic effect.

68. The use of a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogue and combination thereof in the manufacture of a pharmaceutical composition for reducing the development of a metastasis in a patient in need thereof.

69. The use as defined in claim 68, wherein said metastasis is a skeletal metastasis.

70. The use as defined in claim 68, wherein said compound is as defined in SEQ ID NO.:5.

71. The use as defined in claim 68, wherein said compound is a SEQ ID NO.:5 biologically active analogue having an anti-metastatic effect.

72. A method for treating a patient having a metastasis, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

73. A method for treating a patient having a metastatic cancer, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

74. A method for treating a patient having a condition associated with elevated levels of parathyroid hormone-related protein, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

75. A method for treating a patient having PTHrP-induced osteolysis, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.

76. A method for treating a patient having hypercalcemia of malignancy, the method comprising administering to the patient a compound selected from the group consisting of SEQ ID NO.:1, SEQ ID NO.:2, SEQ ID NO.: 3, SEQ ID NO.: 4, SEQ ID NO.:5, SEQ ID NO.:6, biologically active analogues and combinations thereof.
